(19) United States
(12) Patent Application Publication
(10) Pub. No.: US 2025/0381263 A1
(43) Pub. Date: Dec. 18, 2025

(54) Alphavirus (Mayaro virus) Constructs Attenuated for Human and Method of its Use

(71) Applicants: Institut Pasteur de Montevideo, Montevideo (UY); Universidad de la Republica, Montevideo (UY)

(72) Inventors: Gonzalo Moratorio, Montevideo (UY); Pilar Moreno, Montevideo (UY); Alvaro Fajardo, Solymar, Canelones (UY); Diego Simón, Montevideo (UY)

(21) Appl. No.: 19/217,293

(22) Filed: May 23, 2025

Related U.S. Application Data
(60) Provisional application No. 63/651,091, filed on May 23, 2024.

(51) Int. Cl. A61K 39/12; A61K 39/00; A61P 35/00; C12N 7/00

(52) U.S. Cl. CPC A61K 39/12; A61P 35/00; C12N 7/00; A61K 2039/5254; C12N 2770/36134; C12N 2770/36162

(57) ABSTRACT

Genetically engineered alphavirus constructs (e.g., Mayaro virus) attenuated in normal human by increasing CpG dinucleotides frequency and its oncolytic potential against lung and pancreatic cancer. The modified virus may also be used as a live attenuated vaccine against MAYV.

### CROSS-REFERENCE TO RELATED APPLICATION

This application claims the benefit of priority to U.S. Provisional Patent Application No. 63/651,091 filed on May 23, 2024, the entirety of which is incorporated herein by reference

### BACKGROUND

Mayaro virus (MAYV) is an arbovirus (arthropod-borne virus) that, like CHIKV, belongs to the genus Alphavirus, within the family Togaviridae. It is an arthritogenic alphavirus that causes a dengue-like syndrome. The viral particle has an icosahedral capsid approximately 70 nm in diameter and a host cell-derived lipid envelope containing heterodimers of the transmembrane glycoproteins E1 and E2. The genetic material of Mayaro virus consists of a single strand of positive-sense RNA of ˜12 kb (referred to as “genomic RNA” in this disclosure) with two open reading frames (one 7 kb and one 4 kb), each encoding a polyprotein, separated by a short non-coding sequence. These two open reading frames encode the structural and non-structural proteins that orchestrate virion assembly and replication dynamics.

Strong suppression of CpG and UpA dinucleotides is evident in most vertebrate RNA viruses, an adaptation that reflects host cell transcriptomes and evades mammalian immune sensors. However, CpG suppression is absent in invertebrate mRNA and RNA viruses that exclusively target arthropods. Arthropod-borne viruses (arboviruses), including MAYV, which are transmitted between vertebrate hosts by an invertebrate vector, have dinucleotide underrepresentation in CpG and UpA. In contrast, insect-specific viruses (ISVs) have only UpA underrepresentation. This nuanced interplay of dinucleotide representation explains the evolutionary tension of arboviruses in different cytoplasmic environments. Our investigation involving principal component analysis and multidimensional scaling across 8000 viral genomes, confirmed the consistent CpG and UpA underrepresentation in arboviruses (including MAYV).

Oncolytic viruses represent an innovative class of biopharmaceutical agents with immense potential in the field of cancer therapy. By selectively targeting cancer cells, oncolytic therapy has the potential to improve treatment outcomes and reduce the toxic side effects that are commonly associated with traditional cancer treatments, such as chemotherapy and radiation therapy. Stimulation of the immune system may increase the effectiveness of treatment and provide long-term protection against cancer. In addition, oncolytic therapy can be used in combination with other treatments, such as chemotherapy and immunotherapy, to enhance their effectiveness.

### SUMMARY

In some embodiments, the present disclosure provides a non-naturally existing attenuated RNA virus. In one aspect, the attenuated RNA virus is derived from an alpha virus. In another aspect, the attenuated RNA virus is derived from an arbovirus (arthropod-borne virus), or a Mayaro virus (MAYV).

In one aspect, the attenuated RNA virus comprises a plurality of exogenous CpG dinucleotides. In another aspect, the plurality of exogenous CpG dinucleotides is present in the genome of the attenuated RNA virus as a plurality of synonymous mutations, and no single naturally occurring RNA virus contains all exogenous CpG dinucleotides present in the disclosed attenuated RNA virus.

In some embodiments, in the disclosed attenuated RNA virus, the plurality of exogenous CpG dinucleotides are present only at the same positions as those where CpG dinucleotides are present in the genomes of a naturally occurring RNA virus. In one aspect, the plurality of exogenous CpG dinucleotides are present only at the same positions as those at which CpG dinucleotides are present in at least 2% (i.e., at least 80 genomes) of all genomes available to date in the naturally occurring RNA virus database reviewed in the present disclosure.

In some other embodiments, the plurality of exogenous CpG dinucleotides are present only at the same positions as those where CpG dinucleotides exist in at least 5% (i.e., at least 400 genomes) of the 8000 genomes of naturally occurring RNA virus surveyed in the present disclosure. In another aspect, the plurality of exogenous CpG dinucleotides are present only at the same positions as those where CpG dinucleotides exist in at least 20% (i.e., at least 1600 genomes) of the 8000 genomes of naturally occurring RNA virus surveyed in the present disclosure.

In some embodiments, the RNA genome of the disclosed attenuated RNA virus is artificially synthesized. In some other embodiments, the RNA genome of the disclosed attenuated RNA virus is assembled by linking multiple polynucleotide fragments that are artificially synthesized.

In some embodiments, the disclosed attenuated RNA virus has a higher frequency of CpG dinucleotides than the frequency of CpG dinucleotides in a wild-type (i.e., naturally existing) RNA virus of the same origin. In one aspect, the frequency of CpG dinucleotides in the disclosed attenuated RNA virus is 1-500 folds higher than that of a wild-type RNA virus of the same origin. In another aspect, the frequency of CpG dinucleotides in the disclosed attenuated RNA virus is 1-10 folds higher than that of a wild-type RNA virus of the same origin.

In another aspect, the total number of CpG dinucleotides in the disclosed attenuated RNA virus is about 50 to 500 more than that of a wild-type RNA virus of the same origin. In another aspect, the plurality of CpG dinucleotides in the disclosed attenuated RNA virus comprises 1-10000 CpG dinucleotides, or 50-1000, or 100-1000, or 50-300 CpG dinucleotides.

In one embodiment, the plurality of exogenous CpG dinucleotides of the disclosed attenuated RNA virus is present only at positions where CpG dinucleotides are confirmed to exist in 2 or more, 10 or more, 20 or more, 30 or more, 40 or more, 50 or more, or 60 or more different naturally existing mayaro virus genomes.

In another embodiment, not all of the plurality of exogenous CpG dinucleotides of the disclosed attenuated RNA virus are present in the genome of any single naturally existing alpha virus. In another embodiment, not all of the plurality of exogenous CpG dinucleotides of the disclosed attenuated RNA virus are present in the genome of one single naturally existing mayaro virus.

In one embodiment, the plurality of CpG dinucleotides of the disclosed attenuated RNA virus exists only in structural regions of the genome. In another embodiment, the plurality of CpG dinucleotides of the disclosed attenuated RNA virus exists only in non-structural regions of the genome or in both regions, all across the viral genome.

The detailed positions and substitutions of the CpG modification are shown in Table 1. In one embodiment, the plurality of CpG dinucleotides exist only at 2 or more, 10 or more, 20 or more, 30 or more, 40 or more, 50 or more, or 60 or more positions on the genomic RNA selected from all positions listed under Position (POS) column of Table 1.

In one embodiment, the present disclosure provides a method of generating a live attenuated RNA virus or genome thereof. In one aspect, the method includes: (a) providing an infectious RNA virus or a cDNA clone comprising retrotranscript of genome of the infectious RNA virus; and (b) modifying the RNA genome of the infectious RNA virus or the cDNA clone to obtain the attenuated RNA virus or modified cDNA clone comprising the retrotranscript of the genome of the attenuated RNA virus, wherein the modification in step (b) comprises adding one or more CpG dinucleotides to the RNA genome of the infectious RNA or the retrotranscript of the genome of the RNA virus, wherein addition of the one or more CpG dinucleotides does not alter amino acid sequence of the protein encoded by the RNA genome of the infectious RNA virus or the retrotranscript of the genome of the RNA virus, and wherein the one or more CpG dinucleotides are added only at positions where CpG dinucleotides exist in a naturally existing RNA virus or genome thereof.

In another embodiment, in the disclosed method, the RNA virus is an alpha virus, or an arbovirus (arthropod-borne virus), or a Mayaro virus (MAYV). In another embodiment, not all of the plurality of exogenous CpG dinucleotides in the disclosed method are present in the genome of one single naturally existing alpha virus, or one single naturally existing mayaro virus.

In another embodiment, the modifying step (b) is performed by site-directed mutagenesis. In another embodiment, the CpG dinucleotides are introduced by designing and synthesizing polynucleotides. In another embodiment, the synthesized polynucleotides are assembled to form the genome of the disclosed attenuated RNA virus.

In another embodiment, the attenuated RNA virus is oncolytic.

In another embodiment, a method is provided for treating cancer by administering a composition comprising the disclosed attenuated RNA virus to a subject in need thereof. In one aspect, the composition may be administered by intra-tumor injection or by systemic injection. In another aspect, the attenuated RNA virus is encapsulated in nano-particles coated with anti-tumor antibodies.

In another embodiment, a method of preventing or treating Mayaro virus infection is provided by administering a composition comprising the disclosed attenuated RNA virus to a subject in need thereof.

### INCORPORATION BY REFERENCE

The Sequence Listing information contained in the file entitled “IPDM11-00602812US_SL.xml”, created on May 23, 2025, and having a size of 38 kbytes, is hereby incorporated.

### BRIEF DESCRIPTION OF THE DRAWINGS

FIG. 1 illustrates distribution of CpG frequencies among all MAYV available genomes.

FIG. 2 is a schematic representation of MAYV recoded genomes and the number of synonymous mutations comparing with wild type genome.

FIG. 3 shows infectious viral progeny over time. FIG. 3A: Production of infectious viral progeny over time of wild-type (WT), S+, NS+and FG+MAYV viruses in A549 at MOI=3, FIG. 3B: Production of infectious viral progeny over time of WT, S+, NS+ and FG+ MAYV viruses in C6/369 at MOI-3. HPI, Hours post infection; PFU, Plaque Forming Unit.

FIG. 4 shows that the MAYV FG+ is attenuated in vivo. Virus titers in spleen, liver and muscle of mice Balb/cJ2DPI infected with 1e105 PFU/ml of MAYV WT and MAYV FG+.

FIG. 5 shows MAYVWT and MAYV FG+ preferentially killed human adenocarcinoma Lung cells. At the top of the figure we can observe the viability of human adenocarcinoma lung cell line (A549) and normal lung cells (MRC5) confirmed by crystal violet staining after MAYV WT and MAYV FG+ infection at MOls of 0.1 and 1 for 72 hrs. Live cells were stained violet. The same effect was observed by microscopy.

FIG. 6 shows Cytotoxicity of MAYV against NSCLC cell lines was MOI and time-dependent. The cell viability of MAYV-infected A549, H1975 and H838 cells was observed using the MTT Cell Viability Assay at the indicated MOls and times. (A-C) The MOI-dependent cytotoxicity of MAYV against A549, H1975 and H838 cells was confirmed by setting MOI=0.01, 0.1, 1 for 72 hrs. (D-F) The time-dependent cytotoxicity of MAYV against A549, H1975 and H838 cells was confirmed by setting time=24, 48, and 72 h with MOI=0.01, 0.1 and 1.

FIG. 7 shows that MAYV FG+ killed human metastasic breast cancer cells and human pancreatic cancer cells. In the figure we can observe the viability of human Metastasic breast cancer cells (MDA157) and pancreatic cancer cells (L3.6 and Panc-1) confirmed by crystal violet staining after MAYV FG+ infection at MOls of 0.1, 1 and 10 for 72 hrs.

FIG. 8A illustrates that MAYV FG+ infection in NSG mice does not impact in body weight in the treated animals, suggesting that MAYV FG+ was not toxic in vivo at the administered doses

FIG. 8B illustrates the reduction in tumor growth in cell derived Xenograft (CDX) tumor model in NSG mice following intratumoral infection. Tumors were generated by subcutaneous injection of 1×10⁵ A549 cells (left) or 1×10⁶ Panc-1 cells (right). Once tumors reached a size of 120 mm², they were infected with 1×10⁶ PFU of MAYV FG+. Tumor volumes were recorded every two days. Vehicle or virus treatment was administered on days 0, 2, 4, and 6.

FIG. 9 illustrates improved survival of NSG mice with CDX tumors following intratumoral infection with MAYV FG+ . Kaplan-Meier survival curves show the extended survival of mice with treated tumors (A549 CDX in left panel, Panc-1 CDX in right panel) compared to controls

FIG. 10 shows how intratumoral MAYV FG+ infection inhibits tumor growth in a patient-derived xenograft tumor (PDX) compared to the control mice. Male NSG mice received intratumoral administration of 1×10⁶ PFU of MAYV FG+ in 100 μL at the time points indicated by black arrows. Additional doses on day 20 were administered. Tumor growth is expressed as a percentage relative to day 1.

FIG. 11 shows Kaplan-Meier survival curves which demonstrate enhanced survival in male NSG mice bearing patient-derived xenograft (PDX) tumors following intratumoral treatment with MAYV FG+.

FIG. 12 shows examples of cDNA sequences corresponding to the FG+ (SEQ ID NO:1), S+ (SEQ ID NO:2), and NS+ (SEQ ID NO:3) RNA genomes.

### DETAILED DESCRIPTION

In one embodiment, the present disclosure involves computational design and synthetic biology to rationally modify the CpG frequency of MAYV without altering the amino acid sequence.

It has been demonstrated that CpG motif enables the activation of antiviral defenses, primarily mediated by ZAP, which targets non-self RNA for degradation.

Because of the replication defects conferred by CpG introduction into viral genomes, CpG enrichment has been widely proposed as a potential strategy for the development of live attenuated vaccines (Sharp et al 2023; Burns et al 2009, Antzin-Anduetza 2017; Trus et al 2020, Fros et al 2017). However, in all the above cases, the re-coding of the viral genomes was performed without taking into account the natural presence of these newly introduced codons.

The presently disclosed process distinguishes itself by recoding viral genomes based on pre-existing natural variations. By leveraging naturally occurring CpG motifs, the present invention harnesses the power of evolution's own solutions for viral adaptation. This methodology ensures the development of stable genomes with minimal risk of reversion, thus providing a novel and promising avenue for the field of viral genome modification.

The presently disclosed method is not only innovative but also bioinformatically sound, as it maintains the integrity of the viral genome, including protein sequences and functional elements. This design helps mitigate the risks associated with previous recoding techniques while enhancing the potential for creating safer and more effective live attenuated vaccines. Additionally, this approach aligns with the principles of evolutionary biology, as the designed scheme works within the constraints of natural genomic variability to yield more predictable and reliable outcomes in vaccine development.

In another embodiment, few alphaviruses have been identified as oncolytic demonstrating natural tumor targeting and specific replication in tumor cells leading to their death without affecting normal cells. The M1 alphavirus possesses natural oncolytic activity (Zhang et al., 2021), Sindbis virus (SIN) has showed natural tumor targeting (Tseng et al., 2004), and Semliki Forest Virus (SFV). The present invention aims to use another member of the alphaviridae family: Mayaro Virus.

### Definitions

The following definitions are provided to facilitate an understanding of the present disclosure:

The singular forms “a,” “an,” and “the” include plural referents unless the context clearly dictates otherwise.

“Polynucleotide” or “Nucleic acid” or a “nucleic acid molecule” as used herein refers to any DNA or RNA molecule, either single or double stranded and, if single stranded, the molecule of its complementary sequence in either linear or circular form.

As used herein, the term “non-naturally existing” means a composition, or a virus that does not exist in nature and is only known or come into existence through the genetic engineering.

The term “exogenous” means not from within. For purpose of this disclosure, “an exogenous CpG” is one that does not naturally exist in a virus at the specific nucleotide position in the genome of the virus, but instead is introduced through genetic engineering into the virus at that specific position.

The term “attenuated virus” means a virus that is created or modified by reducing its virulence, but is otherwise viable (or “live”).

The term “frequency of CpG dinucleotides” means the total number of CpG dinucleotides per kb of polynucleotide.

The instant disclosure is further illustrated by the following Items:

Item 1: A non-naturally existing attenuated RNA virus, comprising a plurality of exogenous CpG dinucleotides on a genomic RNA, said plurality of exogenous CpG dinucleotides being present in genome of the attenuated RNA virus as a plurality of synonymous mutations, wherein no single naturally occurring RNA virus comprises all exogenous CpG dinucleotides present at the same positions on the genomic RNA as in said attenuated RNA virus.
Item 2. The attenuated RNA virus of Item 1, wherein the plurality of exogenous CpG dinucleotides are present only at the same positions on the genomic RNA as those positions where CpG dinucleotides exist in different genomes of naturally occurring RNA virus.
Item 3. The attenuated RNA virus of any preceding Items, wherein the attenuated RNA virus has a higher frequency of CpG dinucleotides than that of a wild-type RNA virus of same origin.
Item 4. The attenuated RNA virus of any preceding Items, wherein the plurality of CpG dinucleotides comprises 2-1000 CpG dinucleotides, or 50-300 CpG dinucleotides.

Item 5. The attenuated RNA virus of any preceding Items, wherein the RNA virus is an alpha virus, or more particularly, an arbovirus (arthropod-borne virus).

Item 6. The attenuated RNA virus of any preceding Items, wherein the RNA virus is a Mayaro virus (MAYV).
Item 7. The attenuated RNA virus of any preceding Items, wherein the plurality of exogenous CpG dinucleotides is present only at positions where CpG dinucleotides exist in 2 or more, 10 or more, 20 or more, 30 or more, 40 or more, 50 or more, or 60 or more naturally existing Mayaro viruses or genomes thereof.
Item 8. The attenuated RNA virus of any preceding Items, wherein the plurality of CpG dinucleotides exist only at 2 or more, 10 or more, 20 or more, 30 or more, 40 or more, 50 or more, or 60 or more positions on the genomic RNA selected from all positions listed under Position (POS) column of Table 1.
Item 9. The attenuated RNA virus of any preceding Items, wherein the plurality of CpG dinucleotides exist only in structural regions of the genome, or only in non-structural regions of the genome, or in both regions all across the viral genome.
Item 10. The attenuated RNA virus of any preceding Items, wherein the attenuated RNA virus is oncolytic.
Item 11. A method of generating a live attenuated RNA virus or genome thereof, comprising:
(a) providing an infectious RNA virus or a cDNA clone comprising retro-transcript of genome of the infectious RNA virus; and
(b) modifying the RNA genome of the infectious RNA virus or the cDNA clone to obtain the attenuated RNA virus or modified cDNA clone comprising the retrotranscript of the genome of the attenuated RNA virus,
wherein the modification in step (b) comprises adding one or more CpG dinucleotides to the RNA genome of the infectious RNA or the retrotranscript of the genome of the RNA virus, wherein addition of the one or more CpG dinucleotides does not alter amino acid sequence of protein encoded by the RNA genome of the infectious RNA virus or the retrotranscript of the genome of the RNA virus, and wherein the one or more CpG dinucleotides are added only at positions where CpG dinucleotides exist in a naturally existing RNA virus or genome thereof.
   Item 12. The method of Item 11, wherein the RNA virus is an alpha virus, or an arbovirus (arthropod-borne virus), or a Mayaro virus (MAYV).
   Item 13. The method of any of Items 11-12, wherein the one or more CpG dinucleotides are introduced only at positions where CpG dinucleotides are confirmed to exist in at least 10, or at least 20, or at least 30, or at least 40, or at least 50, or at least 60 naturally existing RNA viruses or genomes thereof.
   Item 14. The method of any of Items 11-13, wherein the modifying step is performed by site-directed mutagenesis.
   Item 15. The method of any of Items 11-14, wherein the obtained RNA virus or genome thereof comprises CpG dinucleotides that exist at 2 or more, 10 or more, 20 or more, 30 or more, 40 or more, 50 or more, or 60 or more positions on the genomic RNA selected from positions listed under Position (POS) column of Table 1.
   Item 16. An attenuated RNA virus obtained through the method of any of Items 11-15.
   Item 17. A method of treating of preventing cancer by administering to a subject in need thereof a composition comprising the attenuated RNA virus of any of Items 1-10.
   Item 18. The method of Item 17, wherein the composition is administered by intra-tumor injection or by systemic injection.
   Item 19. The method of any of Items 17-18, wherein the attenuated RNA virus is encapsulated in nano-particles coated with anti-tumor antibodies.
   Item 20. A method of preventing or treating Mayaro virus infection by administering to a subject in need thereof a composition comprising the attenuated RNA virus of any of Items 1-10.
   Item 21. The attenuated RNA virus of any of Items 1-10, wherein the cDNA sequence corresponding to the RNA genome of the attenuated RNA virus shares at least 90%, or at least 95%, or at least 99%, or 100% sequence identity to a sequence selected from the group consisting of SEQ ID No: 1, SEQ ID No: 2 and SEQ ID No: 3. 18

### EXAMPLES

### Example 1 Generation of Different MAYV Synthetic Viruses

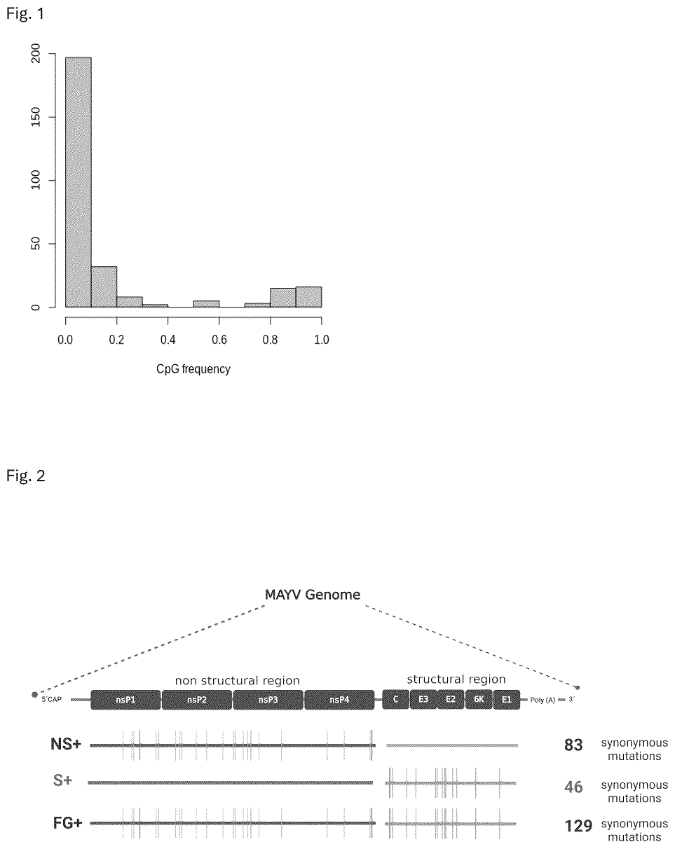

To test the initial concept, 100 natural MAYV genomes were analyzed and CpGs that were present in at least 5% of them to their MAYV infectious clone were added (see FIG. 1).

MAYV infectious clone was then engineered to generate three different MAYV synthetic virus lines which had increased CpG frequency in structural, non-structural, or full genome regions, respectively (see FIG. 2 and table 1).
1. Structural Region CpG overrepresentation (S+)—CpG frequency enhancement only in structural regions.
2. Non-Structural Region CpG overrepresentation (NS+)—CpG frequency enhancement only in non-structural regions.
3. Full Genome CpG overrepresentation (FG+)—CpG frequency increase throughout the genome.

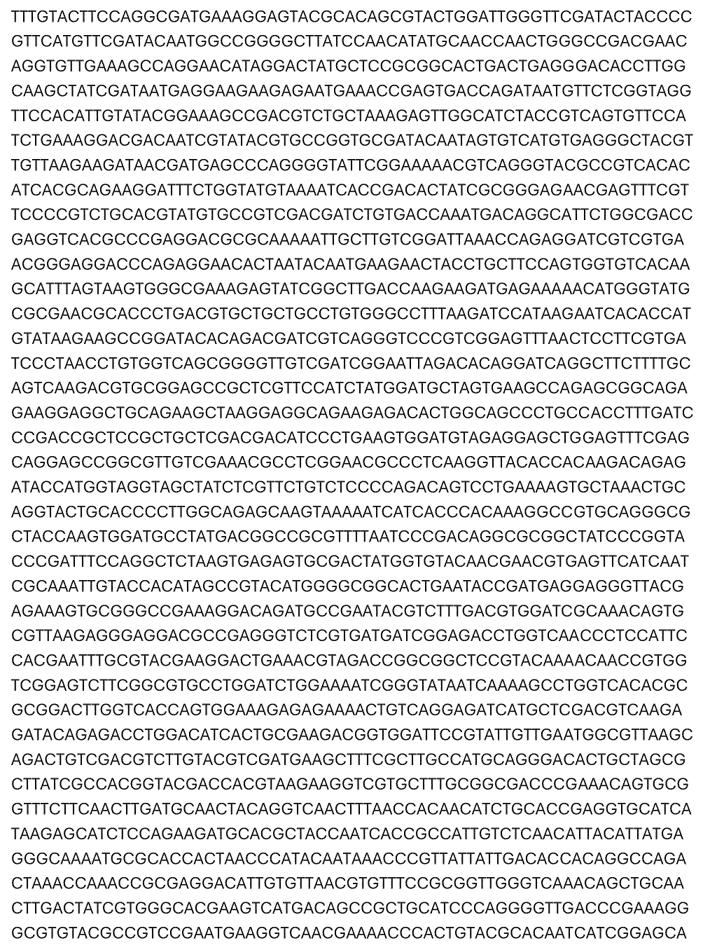

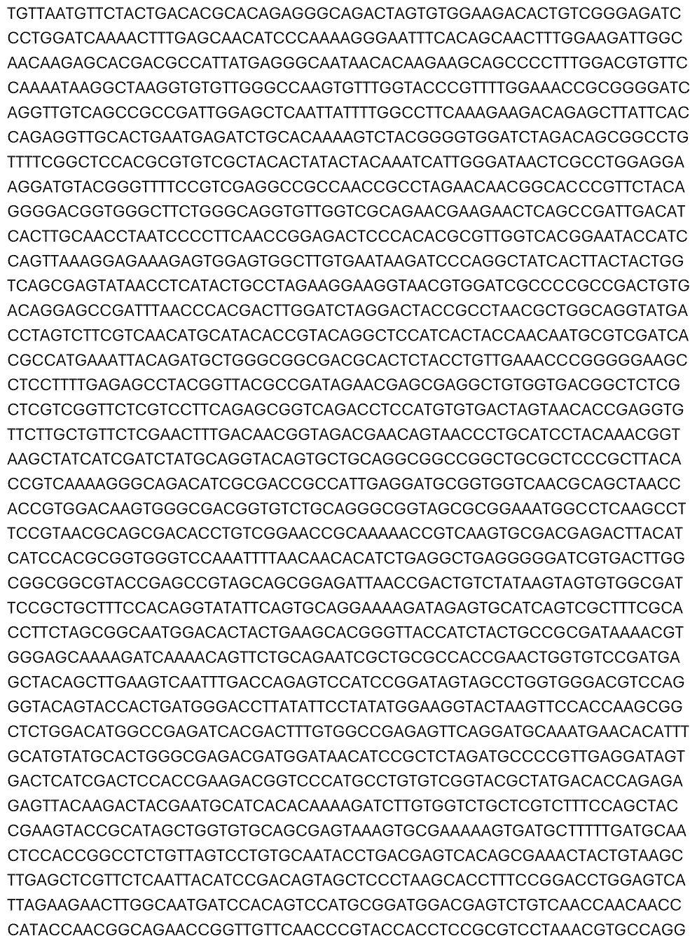

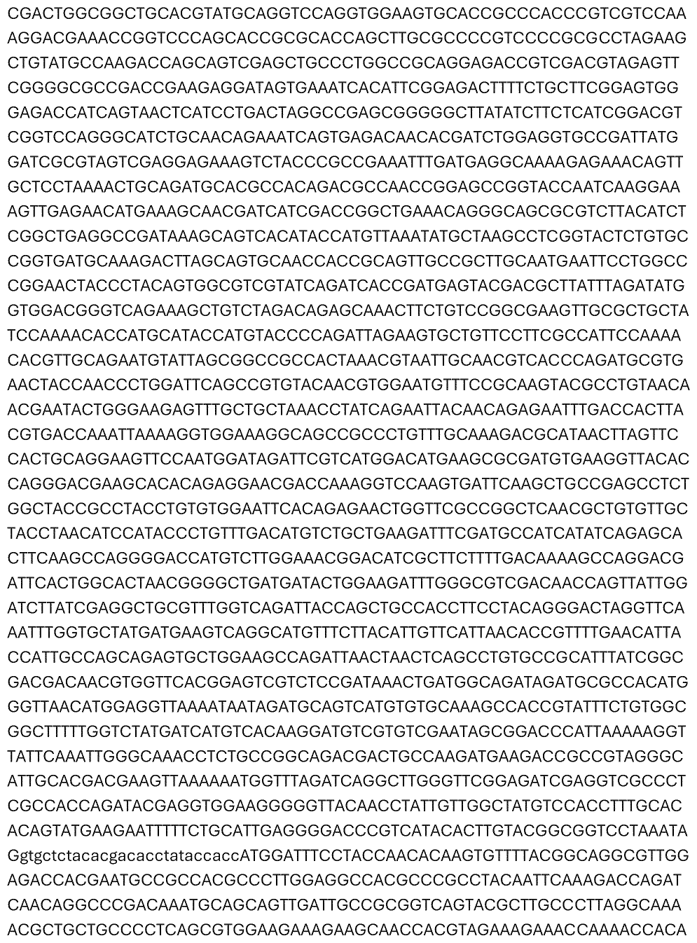

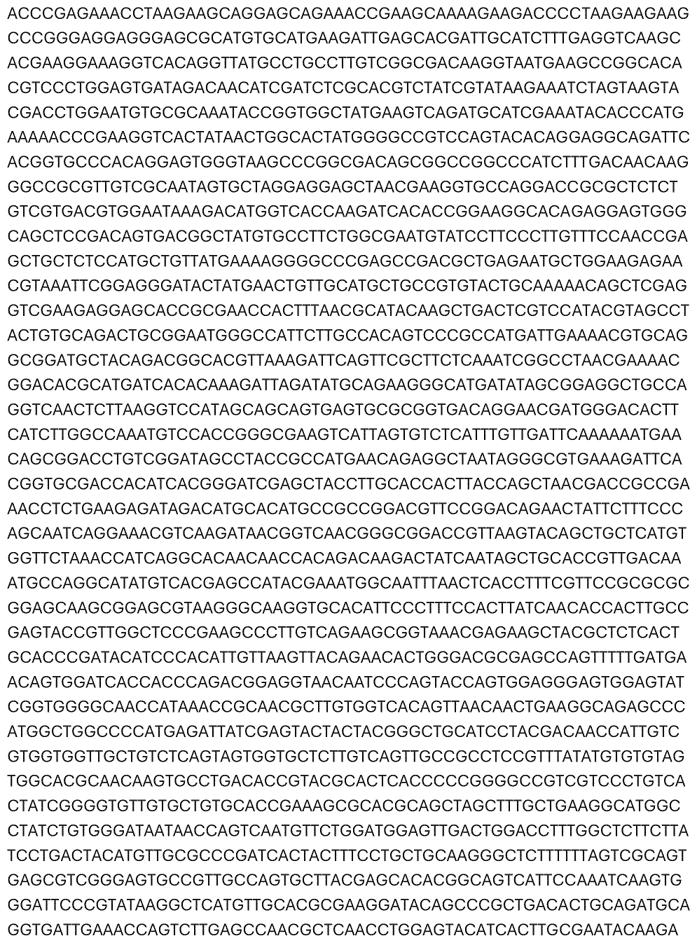

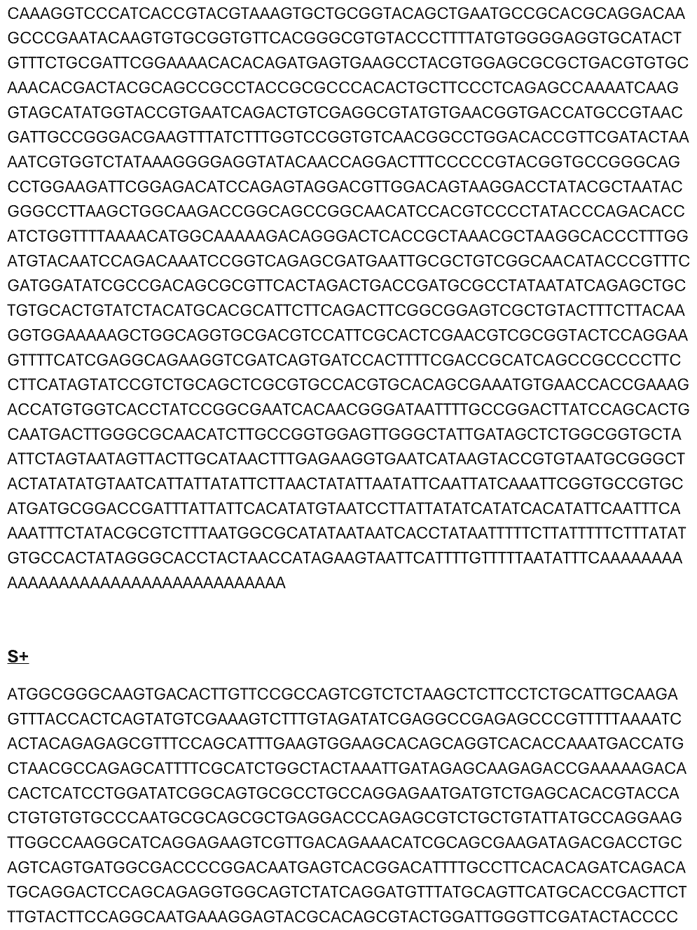

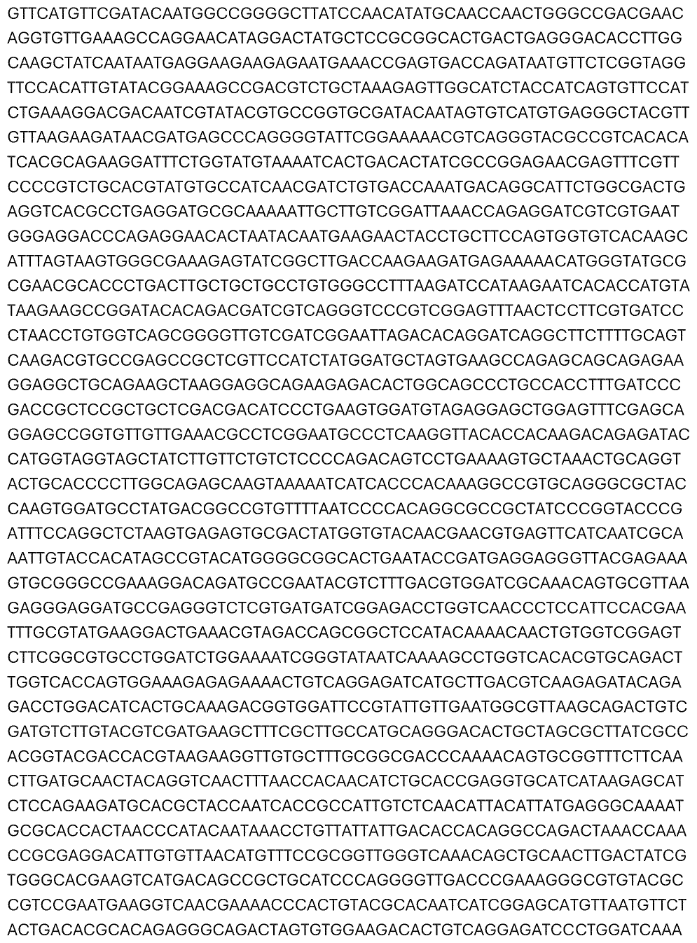

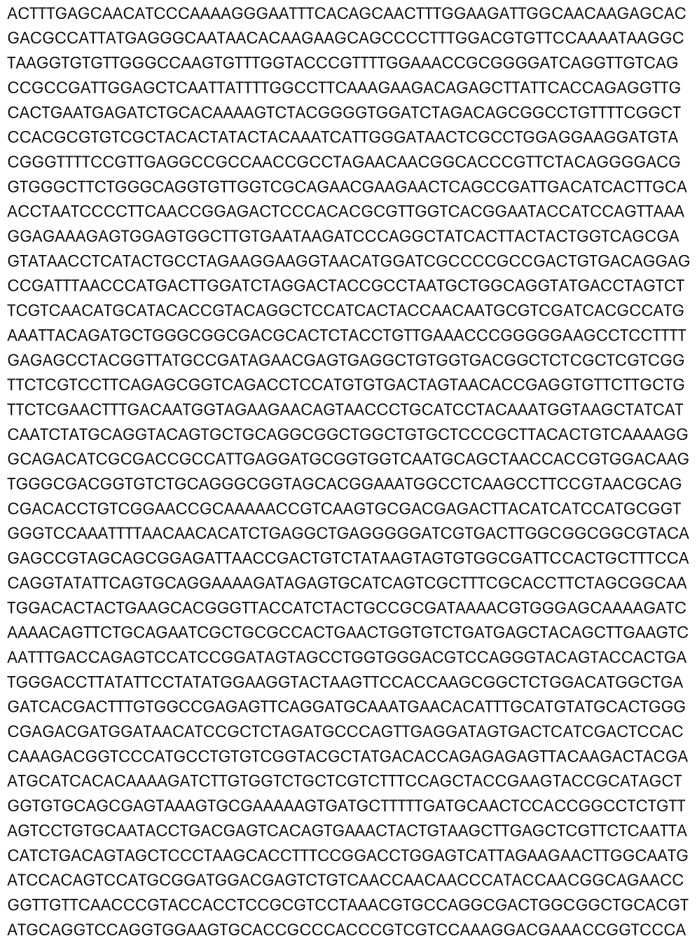

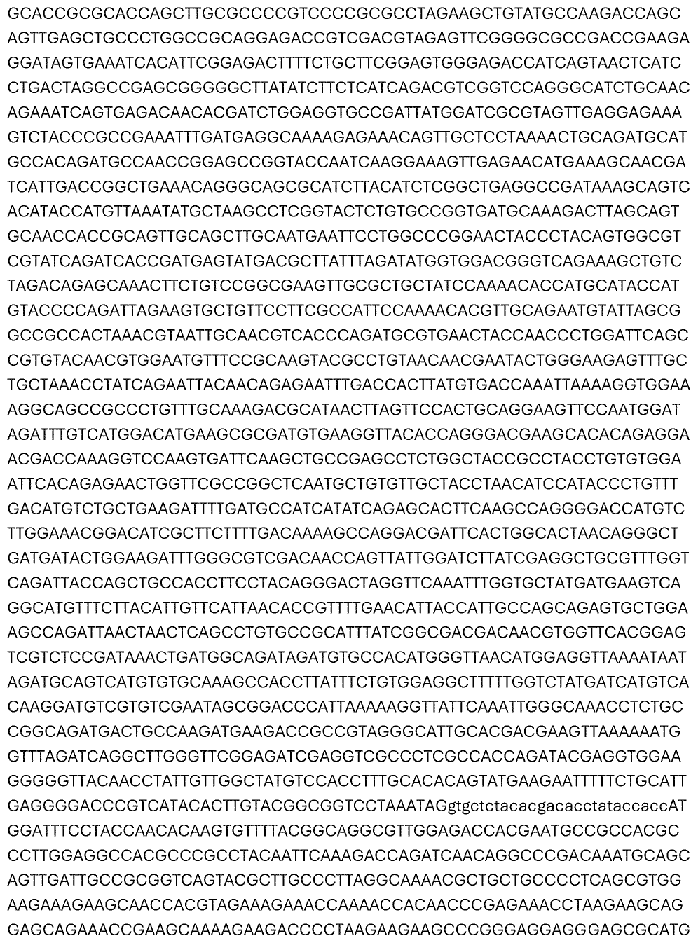

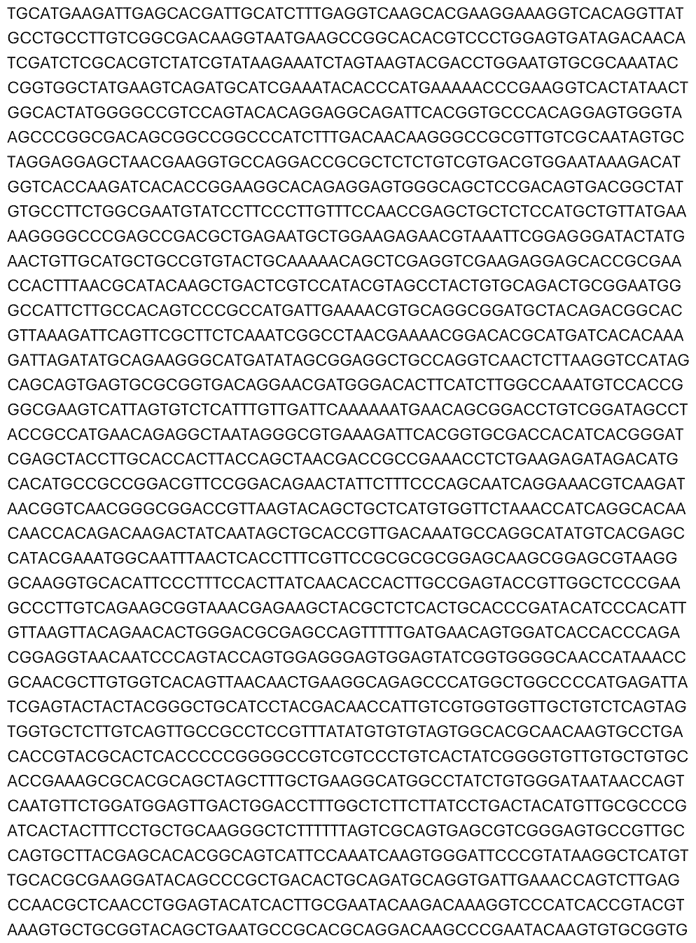

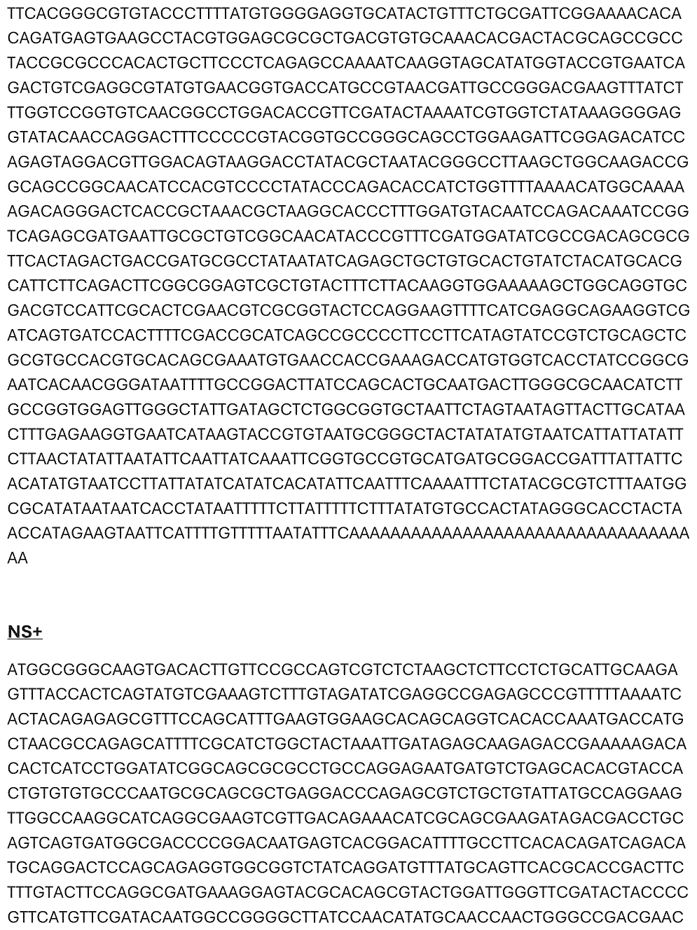

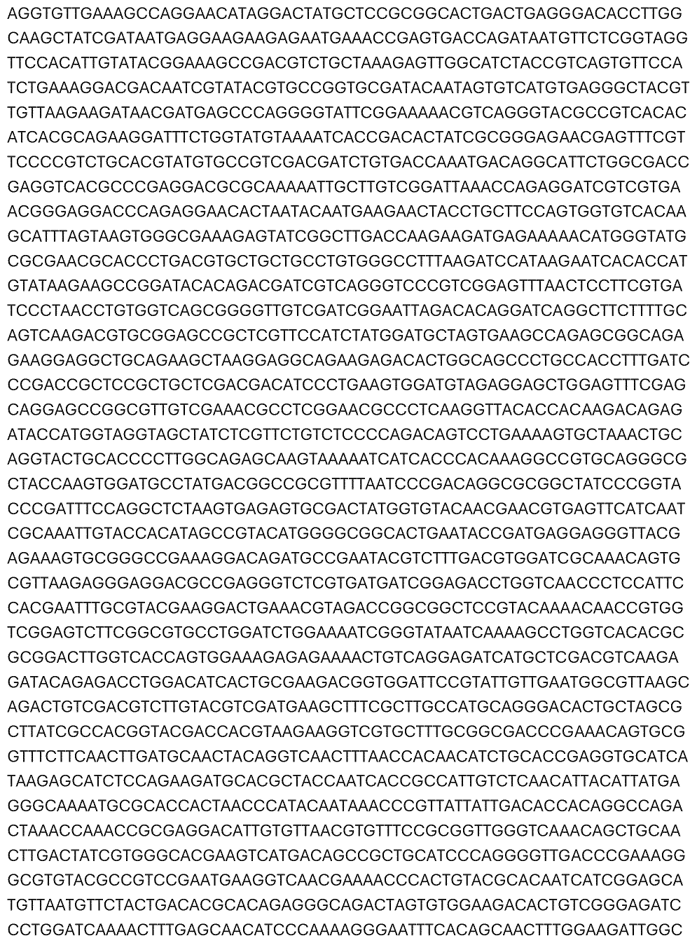

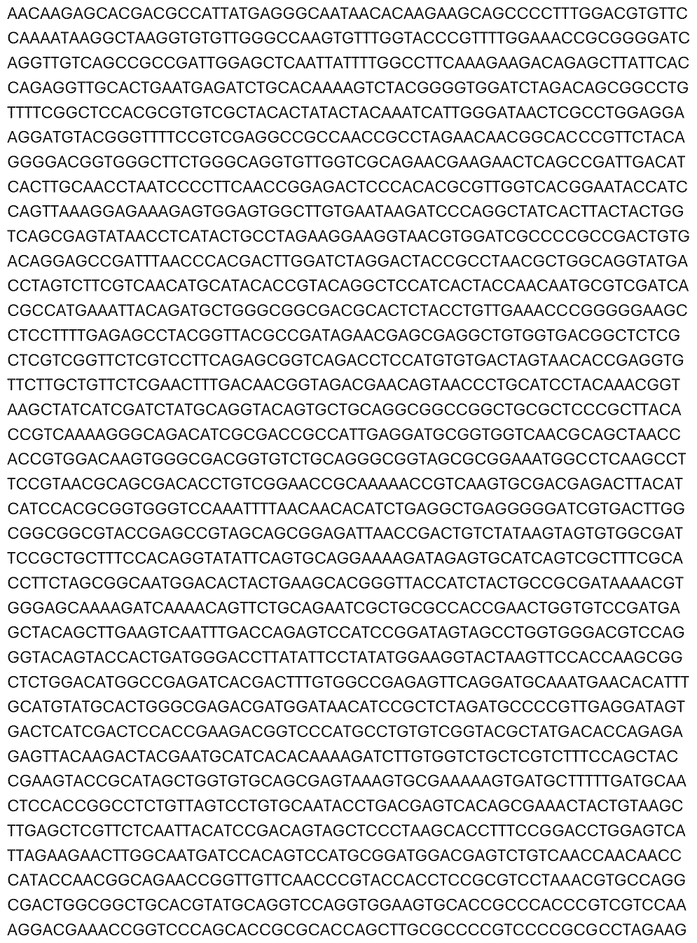

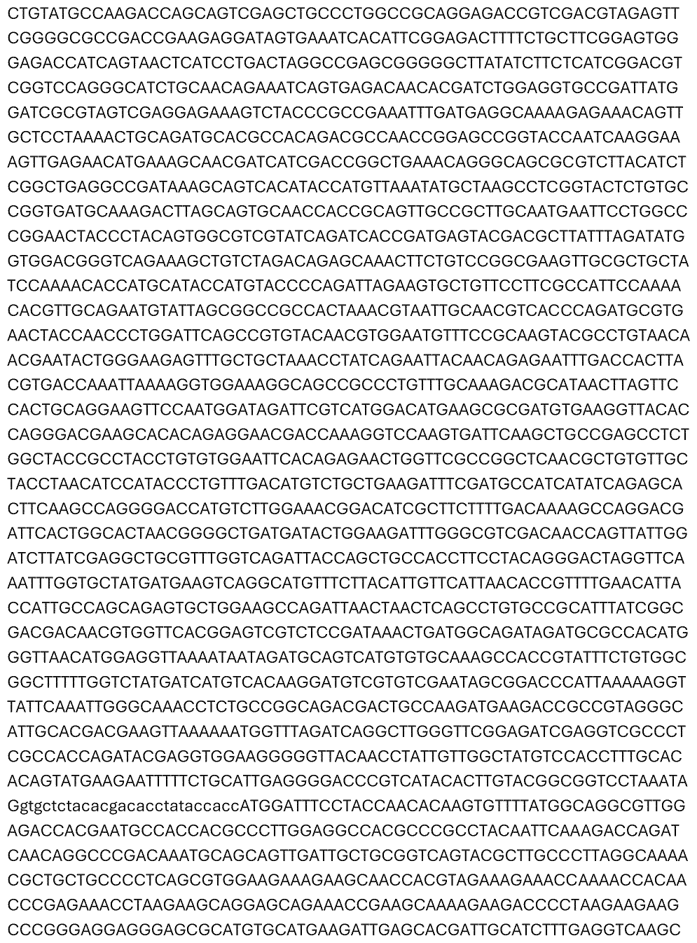

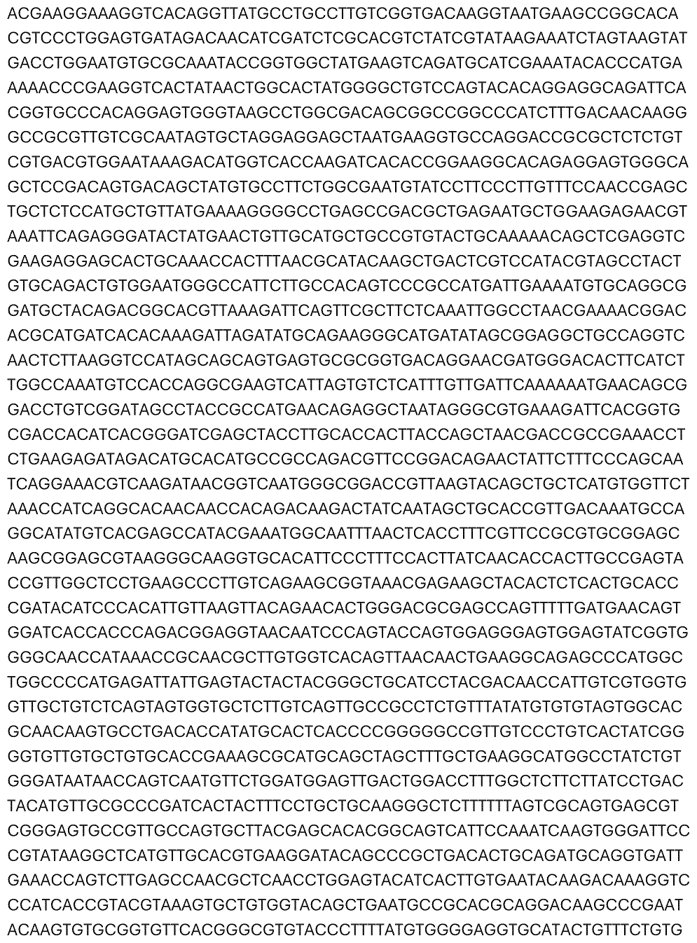

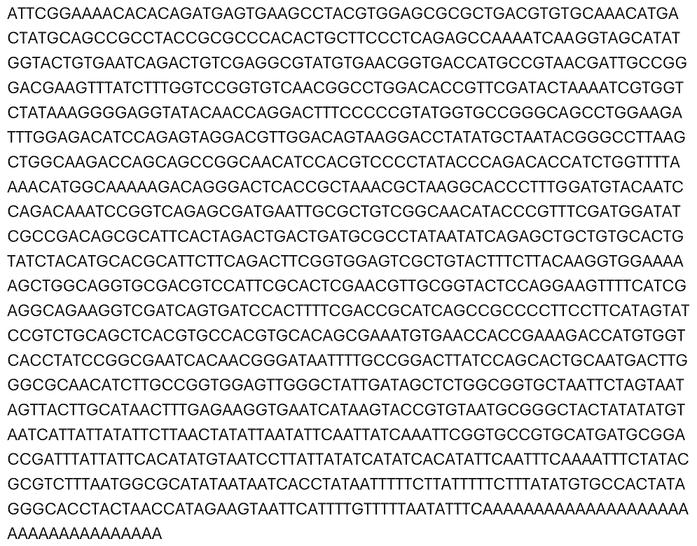

FIG. 12 shows the sequences of the cDNA corresponding to the S+, NS+ and FG+ RNA genomes.

TABLE 1

| POS | [symbol] | S+ | NS+ | FG+ |
|---|---|---|---|---|
| [symbol] | T | T | C | C |
| [symbol] | A | A | C | C |
| [symbol] | A | A | G | G |
| [symbol] | T | T | C | C |
| [symbol] | A | A | G | G |
| [symbol] | A | A | G | G |
| [symbol] | A | A | G | G |
| [symbol] | T | T | C | C |
| [symbol] | C | C | G | G |
| [symbol] | A | A | G | G |
| [symbol] | A | A | G | G |
| [symbol] | T | T | C | C |
| [symbol] | T | T | C | C |
| [symbol] | T | T | C | C |
| [symbol] | T | T | C | C |
| [symbol] | T | T | G | G |
| [symbol] | C | C | G | G |
| [symbol] | A | A | G | G |
| [symbol] | T | T | C | C |
| [symbol] | T | T | C | C |
| [symbol] | T | T | C | C |
| [symbol] | T | T | C | C |
| [symbol] | T | T | C | C |
| [symbol] | C | C | G | G |
| [symbol] | C | C | G | G |
| [symbol] | T | T | C | C |
| [symbol] | T | T | C | C |
| [symbol] | A | A | G | G |
| [symbol] | A | A | G | G |
| [symbol] | T | T | C | C |
| [symbol] | T | T | C | C |
| [symbol] | A | A | G | G |
| [symbol] | T | T | C | C |
| [symbol] | A | A | G | G |
| [symbol] | T | T | C | C |
| [symbol] | T | T | C | C |
| [symbol] | A | A | G | G |
| [symbol] | T | T | C | C |
| [symbol] | A | A | G | G |
| [symbol] | A | A | C | C |
| [symbol] | A | A | G | G |
| [symbol] | T | T | C | C |
| [symbol] | A | A | G | G |
| [symbol] | T | T | C | C |
| [symbol] | T | T | C | C |
| [symbol] | T | T | C | C |
| [symbol] | A | A | G | G |
| [symbol] | T | T | C | C |
| [symbol] | A | A | G | G |
| [symbol] | A | A | C | C |
| [symbol] | A | A | G | G |
| [symbol] | T | T | C | C |
| [symbol] | A | A | G | G |
| [symbol] | T | T | C | C |
| [symbol] | T | T | C | C |
| [symbol] | T | T | C | C |
| [symbol] | T | T | C | C |
| [symbol] | T | T | C | C |
| [symbol] | A | A | C | C |
| [symbol] | T | T | C | C |
| [symbol] | A | A | G | G |
| [symbol] | T | T | C | C |
| [symbol] | T | T | C | C |
| [symbol] | T | T | C | C |
| [symbol] | T | T | C | C |
| [symbol] | A | A | G | G |
| [symbol] | T | T | C | C |
| [symbol] | A | A | C | C |
| [symbol] | A | A | G | G |
| [symbol] | T | T | C | C |
| [symbol] | T | T | C | C |
| [symbol] | T | T | C | C |
| [symbol] | A | A | C | C |
| [symbol] | A | A | G | G |
| [symbol] | T | T | C | C |
| [symbol] | T | T | C | C |
| [symbol] | T | T | C | C |
| [symbol] | A | A | G | G |
| [symbol] | T | T | C | C |
| [symbol] | T | T | C | C |
| [symbol] | T | T | C | C |
| [symbol] | T | T | C | C |
| [symbol] | A | A | G | G |
| [symbol] | A | A | C | C |
| [symbol] | T | T | C | C |
| [symbol] | T | T | C | C |
| [symbol] | T | T | C | C |
| [symbol] | T | T | C | C |
| [symbol] | T | T | C | C |
| [symbol] | A | A | G | G |
| [symbol] | T | T | C | C |
| [symbol] | T | T | G | G |
| [symbol] | A | A | C | C |
| [symbol] | T | C | T | C |
| [symbol] | T | C | T | C |
| [symbol] | A | G | A | G |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | A | G | A | G |
| [symbol] | T | C | T | C |
| [symbol] | A | G | A | G |
| [symbol] | T | C | T | C |
| [symbol] | A | G | A | G |
| [symbol] | T | C | T | C |
| [symbol] | T | C | T | C |
| [symbol] | T | C | T | C |
| [symbol] | A | G | A | G |
| [symbol] | A | G | A | G |
| [symbol] | T | C | T | C |
| [symbol] | T | C | T | C |
| [symbol] | T | C | T | C |
| [symbol] | A | G | A | G |
| [symbol] | T | C | T | C |
| [symbol] | T | C | T | C |
| [symbol] | A | G | A | G |
| [symbol] | T | C | T | C |
| [symbol] | G | C | G | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | T | C | T | C |
| [symbol] | A | G | A | G |
| [symbol] | A | G | A | G |
| [symbol] | T | C | T | C |
| [symbol] | T | C | T | C |
| [symbol] | T | C | T | C |
| [symbol] | A | G | A | G |

[symbol]

indicates data missing or illegible when filed

### Example 2 In vitro and In Vivo Evaluation

The replication kinetics, tissue distribution and virulence/attenuation profiles of these variants were carefully evaluated against MAYV WT in various cell lines of both mammalian and insect origin. Furthermore, the influence of each variant on the replicative capacity of target organs was investigated. Infectivity was assessed using classical plaque assays.

### Example 3 Synthetic MAYVs are Attenuated in Mammalian Cell Lines and Not in Insect Cell Lines

A549 (human lung carcinoma) and C6/36 (Aedes albopictus mosquito larva) cells were infected with the viral stocks of WT, S+, NS+ and FG+ MAYV viruses at an MOI=3. MAYV were collected in clarified supernatant at different post-infection times. Viral titers were determined by plaque assay. Vero-E6 cells were seeded in six-well plates and virus preparations were serially diluted in serum-free DMEM medium. Cells were washed twice with PBS and infected with 250 ul of the dilution for 30 minutes at 37° C., followed by a solid overlay of DMEM medium and 1% wt/vol agarose. Two days after infection, cells were fixed and stained with crystal violet 0.2% and plaques were enumerated.

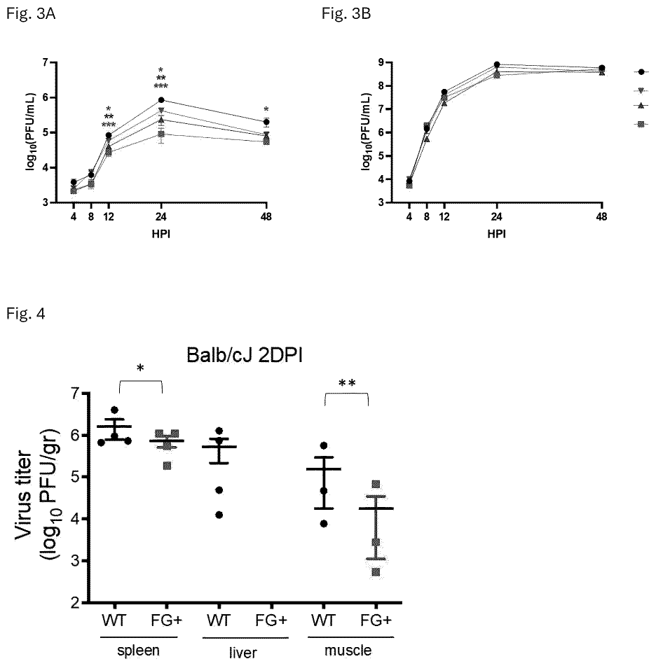

The results showed that MAYV viral titer of CpG mutants S+, NS+ and FG+ are significantly lower at 12 and 24 hr post-infection (HPI) compared to MAYV WT in mammalian cells (A549) (FIG. 3A). However, recoded MAYVs grows as the wild-type virus in insect cells (C6/36) (FIG. 3B). These results confirm the in vitro attenuation of the synthetic viruses in mammalian cells, while showing that there are not artifacts involved, since they replicate well in insect cell lines.

Of these three synthetic viruses, the FG+ was the one which showed a more attenuating effect in cell culture. Because this was the virus that showed the greatest effect and therefore the highest level of safety for the objectives, in vivo studies were 27 continued with this synthetic virus.

### Example 4 MAYV FG+ Synthetic Virus is Attenuated In Vivo

To evaluate this attenuation strategy in vivo, mice were given a dose (1e10⁵ PFU/ml) of wild-type or FG+ and virus titers were determined over 2 days post infection. FG+ virus has significantly lower viral titers in spleen and muscle and lacks replication in liver compared to wild-type. (FIG. 4). These results confirm the in vivo attenuation of the synthetic virus FG+.

### Example 5 Genomic Stability of MAYV Constructs

To analyze the stability of the aggregated CpGs in each MAYV mutant genome, 10 serial infections (or blind passages) were performed in mammalian and insect cell lines (A549 and C6/36) after several infection cycles.

Cell line monolayers were infected with NS+, S+, FG+ and WT virus stocks, the genome sequence of which had already been confirmed, and cultured in the appropriate culture medium for each cell type (DMEM for A549 and Vero and Leibovitz's L-15 for C6/36) containing 2% SFB. At 48 hours post infection, the cell culture medium was collected and clarified by centrifugation at 1000 g for 1 minute. 50 uL of the supernatant was used to infect a new cell monolayer in the same manner as described above. This procedure was repeated 10 times and the supernatant from each blind pass was stored at −80° C.

RNA was extracted from the supernatants of passages 5 and 10 of each virus type (WT, NS+, S+ and FG+). Sequencing was performed using the same protocol as for the viral stocks. The whole genome sequences of NS+, S+, FG+ and WT extracted from blind passages 5 and 10 in each cell type (A549, Vero and C6/36) were compared with those of their respective viral stocks using SeqMan version 7.0 software (Lasergene, DNASTAR, USA). This program allows detection of single nucleotide polymorphisms (SNPs) based on a reference sequence (in this case, the viral stocks).

The results showed that all the aggregated CpGs were retained in the synthetic viruses after the 10 blind passages, indicating their stability after 10 cycles of infection. On the other hand, the WT virus did not acquire any new CpGs. This result confirmed that the genomes of the synthetic virus of the present disclosure have little chance of reversion.

### Example 6 MAYV WT and MAYV FG+ Reaches and Destroy Lung Adenocarcinoma Cancer Cells Without Affecting Normal Cells

To evaluate the oncolytic effect of MAYVWT and MAYVFG+, infections were performed in three different human non-small cell lung cancers (NSCLC) cell lines (A549, H1975 and H838) and one non tumoral lung cell line (MRC-5).

First step was to determine whether MAYVWT and MAYVFG+ infection induced oncolytic destruction of human adenocarcinoma lung cell line (A549) and in normal lung cells (MRC5) by crystal violet staining. Cell lines were infected with MAYVWT and MAYVFG+ at a multiplicity of infection (MOI) of 0.1, and 1 for 1 h, and the virus suspension was replaced with 1 ml of fresh medium. After 72 hours, the cells were fixed with 4% formaldehyde for 30 minutes. The remaining formaldehyde solution was removed and the cells were stained with 0.2% crystal violet for 10 minutes. Finally, the staining solution was removed and washed with distilled water, and the plates were dried for 30 minutes.

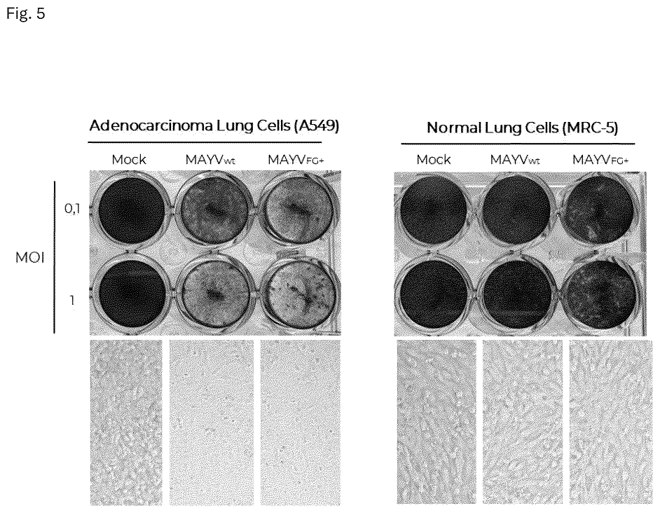

The results showed that MAYVWT and MAYVFG+ infection resulted in extensive oncolytic activity in the A549 cell line but not in the normal lung cell line (FIG. 5). The same effect was observed by microscopy.

### Example 7 MAYV WT Cytotoxicity in NSCLC Cell Lines was Time-Dependent and Concentration-Dependent

Cell viability in NSCLC cell lines (A549, H1975, and H838) was analyzed using the MTT assay to determine whether cytotoxicity due to MAYVWT infection was dependent on MOI, duration of infection, or both.

Briefly, cells were seeded in 96-well plates at 50000 cells per well in 100 μL of medium. After 24 hrs, cells were infected with MAYVWT at MOIs=0.01, 0.1, 1 for 1 h, and the virus suspension was replaced with fresh medium (100u). After 24, 48 and 72 h of infection 3-(4,5-dimethylthiazol-2-yl)-2,5-diphenyltetrazolium bromide (MTT) was added (5 mg/mL), and incubated at 37° C. for 3-4 h. The MTT containing medium was removed, and the formazan crystals were dissolved in DMSO and isopropanol (1v/1v). The optical absorbance was measured at 570 nm using a microplate reader. The infection rate was determined by the formula: Abs sample/Abs mock*100.

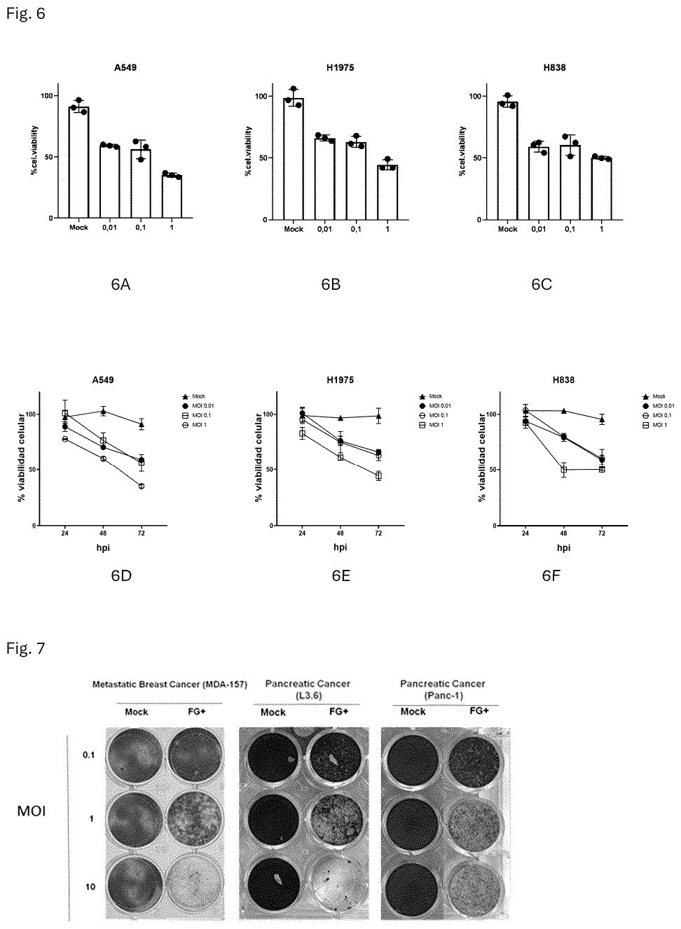

When A549, H1975 and H838 cells were infected with MAYVWT at MOIs=0.01, 0.1, 1, it was found that cytotoxicity at 72 h increased with higher MOls (FIG. 6A-C). Next, when the same cell lines were infected with MAYVWT at MOI of 0,01, 0.1 and 1, and cell viability was evaluated at 24, 48 and 72 hrs. The results show that cytotoxicity increased with time (FIG. 6D-F).

### Example 8 MAYV FG+ Reaches and Destroys Human Metastatic Breast Cancer Cells and Human Pancreatic Cancer Cells

To evaluate the oncolytic effect of MAYVFG+, infections were performed in three different human cancer cell lines: a metastatic breast cancer cell line (MDA-MB-231) and two pancreatic cancer cell lines (L3.6 and Panc-1).

The first step was to determine whether MAYVFG+ infection induced oncolytic destruction in these cancer cells by crystal violet staining. Cell lines were infected with MAYVFG+ at a multiplicity of infection (MOI) of 0.1, 1 and 10 for 1 hour, after which the virus suspension was replaced with 1 ml of fresh medium. After 72 hours, the cells were fixed with 4% formaldehyde for 30 minutes, the fixative was removed, and the cells were stained with 0.2% crystal violet for 10 minutes. Finally, the staining solution was removed, the plates were washed with distilled water, and dried for 30 minutes.

The results showed that MAYVFG+ infection resulted in oncolytic activity in MDA157, L3.6 and Panc-1 cell lines (FIG. 7).

### Example 9

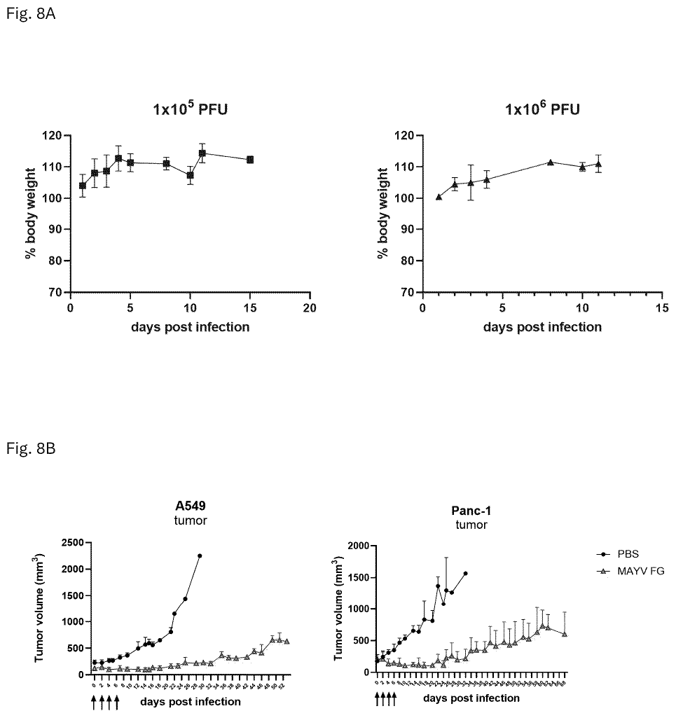

MAYV FG+ infection in NSG mice showed no significant decrease in body weight in the treated animals, suggesting that MAYV FG+ was not toxic in vivo at the administered doses (FIG. 8A). To evaluate the toxicity effect of MAYV FG+ Six-week-old NSG mice (n=3) received a dose of 1×10⁵ or 1×10⁶ PFU in 100 μL via intraperitoneal injection. Mice were weighed every two days, On day 15, all animals were sacrificed. Graph show the percentage of body weight in relation to days post-infection in NSG mice.

### Example 10 Intratumoral Infection with MAYV FG+ Suppresses Tumor Growth and Improves Survival in NSG Mice CDX Tumor Model

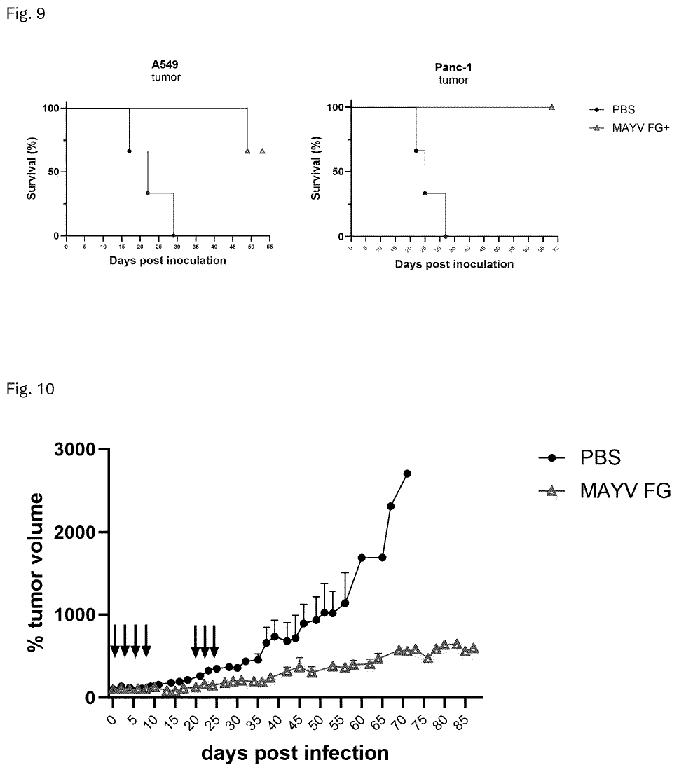

To evaluate the oncolytic effect of MAYV FG+ in vivo, 4-6 weeks old male NSG mice were used. Tumors were generated by subcutaneous injection of 1×10⁵ A549 cells or 1×10⁶ Panc-1 cells (Cell derived Xenograft-CDX). Once tumors reached a size of 120 mm², they were infected with 1×10⁶ PFU of MAYV FG+. Mice received a total of four doses, administered every two days, starting when tumors reached 120 mm³. Tumor growth was monitored by measuring tumor volumes every two days using calipers, and tumor volume was calculated as (length×width²)/2 (FIG. 8B). Kaplan-Meier survival curves show the extended survival of mice with treated tumors (A549 CDX in left panel, Panc-1 CDX in right panel) compared to controls (FIG. 9)

### Example 11 Intratumoral Infection with MAYV FG+ Suppresses Tumor Growth and Improves Survival in NSG Mice PDX Pancreatic Tumor Model

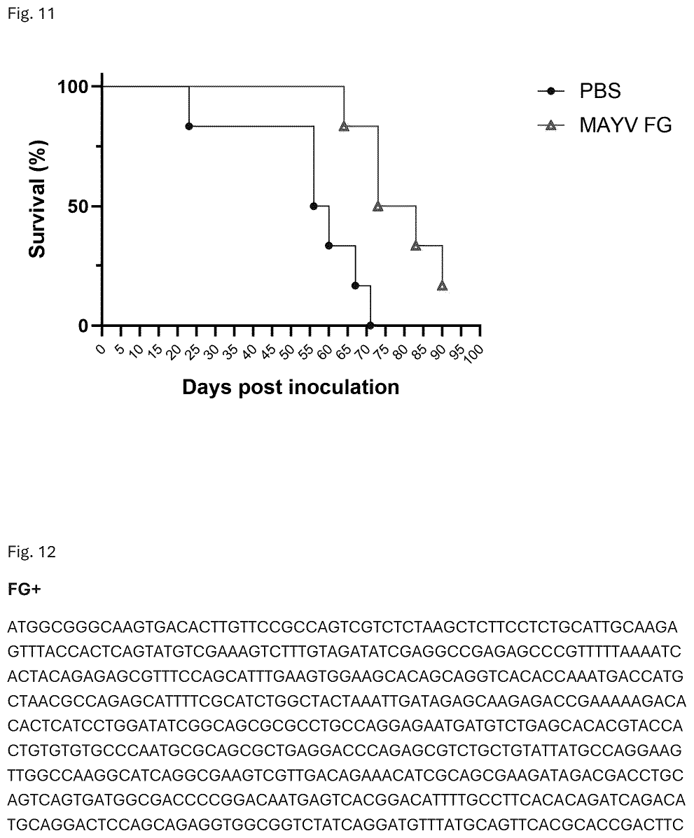

To evaluate the oncolytic effect of MAYV FG+ in PDX tumor model, 4-6 weeks old male NSG mice were used. A 2×2 mm fragment of a patient-derived pancreatic tumor was subcutaneously implanted in the dorsal region of the mice. Once tumors reached a size of 120 mm², they were infected with 1×10⁶ PFU of MAYV FG+. Four doses were administered every two days once the tumor reached 120 mm³, followed by three additional doses 12 days later. Tumor growth was monitored by measuring tumor volumes every two days using calipers, and tumor volume was calculated as (length×width²)/2 (FIG. 10). Kaplan-Meier survival curves show the extended survival of mice with treated pancreatic tumor compared to controls (FIG. 11).

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and compositions described herein may be embodied in a variety of other forms; furthermore, various omissions, substitutions and changes in the form of the methods and compositions described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A non-naturally existing attenuated RNA virus, comprising a plurality of exogenous CpG dinucleotides on a genomic RNA, said plurality of exogenous CpG dinucleotides being present in genome of the attenuated RNA virus as a plurality of synonymous mutations, wherein no single naturally occurring RNA virus comprises all exogenous CpG dinucleotides present at the same positions on the genomic RNA as in said attenuated RNA virus.

2. The attenuated RNA virus of claim 1, wherein the plurality of exogenous CpG dinucleotides are present only at the same positions on the genomic RNA as those positions where CpG dinucleotides exist in different genomes of naturally occurring RNA virus.

3. The attenuated RNA virus of claim 1, wherein the attenuated RNA virus has a higher frequency of CpG dinucleotides than that of a wild-type RNA virus of same origin.

4. The attenuated RNA virus of claim 1, wherein the plurality of CpG dinucleotides comprises 2-1000 CpG dinucleotides, or 50-300 CpG dinucleotides.

5. The attenuated RNA virus of claim 1, wherein the RNA virus is an alpha virus, or more particularly, an arbovirus (arthropod-borne virus).

6. The attenuated RNA virus of claim 1, wherein the RNA virus is a Mayaro virus (MAYV).

7. The attenuated RNA virus of claim 6, wherein the plurality of exogenous CpG dinucleotides is present only at positions where CpG dinucleotides exist in 2 or more, 10or more, 20 or more, 30 or more, 40 or more, 50 or more, or 60 or more naturally existing Mayaro viruses or genomes thereof.

8. The attenuated RNA virus of claim 6, wherein the plurality of CpG dinucleotides exist at 2 or more, 10 or more, 20 or more, 30 or more, 40 or more, 50 or more, or 60 or more positions on the genomic RNA selected from positions listed under Position (POS) column in Table 1.

9. The attenuated RNA virus of claim 6, wherein the plurality of CpG dinucleotides exist only in structural regions of the genome, or only in non-structural regions of the genome, or in both regions all across the viral genome.

10. The attenuated RNA virus of claim 1, wherein the attenuated RNA virus is oncolytic.

11. A method of generating a live attenuated RNA virus or genome thereof, comprising:
(a) providing an infectious RNA virus or a cDNA clone comprising retrotranscript of genome of the infectious RNA virus; and
(b) modifying the RNA genome of the infectious RNA virus or the cDNA clone to obtain the attenuated RNA virus or modified cDNA clone comprising the retrotranscript of the genome of the attenuated RNA virus,
wherein the modification in step (b) comprises adding one or more CpG dinucleotides to the RNA genome of the infectious RNA or the retrotranscript of the genome of the RNA virus, wherein addition of the one or more CpG dinucleotides does not alter amino acid sequence of protein encoded by the RNA genome of the infectious RNA virus or the retrotranscript of the genome of the RNA virus, and wherein the one or more CpG dinucleotides are added only at positions where CpG dinucleotides exist in a naturally existing RNA virus or genome thereof.

12. The method of claim 11, wherein the RNA virus is an alpha virus, or an arbovirus (arthropod-borne virus), or a Mayaro virus (MAYV).

13. The method of claim 11, wherein the one or more CpG dinucleotides are introduced only at positions where CpG dinucleotides are confirmed to exist in at least 10, or at least 20, or at least 30, or at least 40, or at least 50, or at least 60, naturally existing RNA viruses or genomes thereof.

14. The method of claim 11, wherein the modifying step is performed by site-directed mutagenesis.

15. The method of claim 12, wherein the obtained RNA virus or genome thereof comprises CpG dinucleotides that exist at 2 or more, 10 or more, 20 or more, 30 or more, 40 or more, 50 or more, or 60 or more positions on the genomic RNA selected from positions listed under Position (POS) column of Table 1.

16. An attenuated RNA virus obtained through the method of claim 11.

17. A method of treating or preventing cancer by administering to a subject in need thereof a composition comprising the attenuated RNA virus of claim 10.

18. The method of claim 17, wherein the composition is administered by intra-tumor injection or by systemic injection.

19. The method of claim 17, wherein the attenuated RNA virus is encapsulated in nano-particles coated with anti-tumor antibodies.

20. A method of preventing or treating Mayaro virus infection by administering to a subject in need thereof a composition comprising the attenuated RNA virus of claim 1.

21. The attenuated RNA virus of claim 1, wherein the cDNA sequence corresponding to the RNA genome of the attenuated RNA virus shares at least 90%, or at least 95%, or at least 99%, or 100% sequence identity to a sequence selected from the group consisting of SEQ ID No: 1, SEQ ID No: 2 and SEQ ID No: 3.


SEQUENCE LISTING (3 sequences)

SEQ ID NO: 1 - DNA, 11462 bp, synthetic construct
  source 1..11462: mol_type = other DNA
atggcgggcaagtgacacttgttccgccagtcgtctctaagctcttcctctgcattgcaagagtttaccactcagtatgtcgaaagtctttgtagatatcgaggccgagagcccgtttttaaaatcactacagagagcgtttccagcatttgaagtggaagcacagcaggtcacaccaaatgaccatgctaacgccagagcattttcgcatctggctactaaattgatagagcaagagaccgaaaaagacacactcatcctggatatcggcagcgcgcctgccaggagaatgatgtctgagcacacgtaccactgtgtgtgcccaatgcgcagcgctgaggacccagagcgtctgctgtattatgccaggaagttggccaaggcatcaggcgaagtcgttgacagaaacatcgcagcgaagatagacgacctgcagtcagtgatggcgaccccggacaatgagtcacggacattttgccttcacacagatcagacatgcaggactccagcagaggtggcggtctatcaggatgtttatgcagttcacgcaccgacttctttgtacttccaggcgatgaaaggagtacgcacagcgtactggattgggttcgatactaccccgttcatgttcgatacaatggccggggcttatccaacatatgcaaccaactgggccgacgaacaggtgttgaaagccaggaacataggactatgctccgcggcactgactgagggacaccttggcaagctatcgataatgaggaagaagagaatgaaaccgagtgaccagataatgttctcggtaggttccacattgtatacggaaagccgacgtctgctaaagagttggcatctaccgtcagtgttccatctgaaaggacgacaatcgtatacgtgccggtgcgatacaatagtgtcatgtgagggctacgttgttaagaagataacgatgagcccaggggtattcggaaaaacgtcagggtacgccgtcacacatcacgcagaaggatttctggtatgtaaaatcaccgacactatcgcgggagaacgagtttcgttccccgtctgcacgtatgtgccgtcgacgatctgtgaccaaatgacaggcattctggcgaccgaggtcacgcccgaggacgcgcaaaaattgcttgtcggattaaaccagaggatcgtcgtgaacgggaggacccagaggaacactaatacaatgaagaactacctgcttccagtggtgtcacaagcatttagtaagtgggcgaaagagtatcggcttgaccaagaagatgagaaaaacatgggtatgcgcgaacgcaccctgacgtgctgctgcctgtgggcctttaagatccataagaatcacaccatgtataagaagccggatacacagacgatcgtcagggtcccgtcggagtttaactccttcgtgatccctaacctgtggtcagcggggttgtcgatcggaattagacacaggatcaggcttcttttgcagtcaagacgtgcggagccgctcgttccatctatggatgctagtgaagccagagcggcagagaaggaggctgcagaagctaaggaggcagaagagacactggcagccctgccacctttgatcccgaccgctccgctgctcgacgacatccctgaagtggatgtagaggagctggagtttcgagcaggagccggcgttgtcgaaacgcctcggaacgccctcaaggttacaccacaagacagagataccatggtaggtagctatctcgttctgtctccccagacagtcctgaaaagtgctaaactgcaggtactgcaccccttggcagagcaagtaaaaatcatcacccacaaaggccgtgcagggcgctaccaagtggatgcctatgacggccgcgttttaatcccgacaggcgcggctatcccggtacccgatttccaggctctaagtgagagtgcgactatggtgtacaacgaacgtgagttcatcaatcgcaaattgtaccacatagccgtacatggggcggcactgaataccgatgaggagggttacgagaaagtgcgggccgaaaggacagatgccgaatacgtctttgacgtggatcgcaaacagtgcgttaagagggaggacgccgagggtctcgtgatgatcggagacctggtcaaccctccattccacgaatttgcgtacgaaggactgaaacgtagaccggcggctccgtacaaaacaaccgtggtcggagtcttcggcgtgcctggatctggaaaatcgggtataatcaaaagcctggtcacacgcgcggacttggtcaccagtggaaagagagaaaactgtcaggagatcatgctcgacgtcaagagatacagagacctggacatcactgcgaagacggtggattccgtattgttgaatggcgttaagcagactgtcgacgtcttgtacgtcgatgaagctttcgcttgccatgcagggacactgctagcgcttatcgccacggtacgaccacgtaagaaggtcgtgctttgcggcgacccgaaacagtgcggtttcttcaacttgatgcaactacaggtcaactttaaccacaacatctgcaccgaggtgcatcataagagcatctccagaagatgcacgctaccaatcaccgccattgtctcaacattacattatgagggcaaaatgcgcaccactaacccatacaataaacccgttattattgacaccacaggccagactaaaccaaaccgcgaggacattgtgttaacgtgtttccgcggttgggtcaaacagctgcaacttgactatcgtgggcacgaagtcatgacagccgctgcatcccaggggttgacccgaaagggcgtgtacgccgtccgaatgaaggtcaacgaaaacccactgtacgcacaatcatcggagcatgttaatgttctactgacacgcacagagggcagactagtgtggaagacactgtcgggagatccctggatcaaaactttgagcaacatcccaaaagggaatttcacagcaactttggaagattggcaacaagagcacgacgccattatgagggcaataacacaagaagcagcccctttggacgtgttccaaaataaggctaaggtgtgttgggccaagtgtttggtacccgttttggaaaccgcggggatcaggttgtcagccgccgattggagctcaattattttggccttcaaagaagacagagcttattcaccagaggttgcactgaatgagatctgcacaaaagtctacggggtggatctagacagcggcctgttttcggctccacgcgtgtcgctacactatactacaaatcattgggataactcgcctggaggaaggatgtacgggttttccgtcgaggccgccaaccgcctagaacaacggcacccgttctacaggggacggtgggcttctgggcaggtgttggtcgcagaacgaagaactcagccgattgacatcacttgcaacctaatccccttcaaccggagactcccacacgcgttggtcacggaataccatccagttaaaggagaaagagtggagtggcttgtgaataagatcccaggctatcacttactactggtcagcgagtataacctcatactgcctagaaggaaggtaacgtggatcgccccgccgactgtgacaggagccgatttaacccacgacttggatctaggactaccgcctaacgctggcaggtatgacctagtcttcgtcaacatgcatacaccgtacaggctccatcactaccaacaatgcgtcgatcacgccatgaaattacagatgctgggcggcgacgcactctacctgttgaaacccgggggaagcctccttttgagagcctacggttacgccgatagaacgagcgaggctgtggtgacggctctcgctcgtcggttctcgtccttcagagcggtcagacctccatgtgtgactagtaacaccgaggtgttcttgctgttctcgaactttgacaacggtagacgaacagtaaccctgcatcctacaaacggtaagctatcatcgatctatgcaggtacagtgctgcaggcggccggctgcgctcccgcttacaccgtcaaaagggcagacatcgcgaccgccattgaggatgcggtggtcaacgcagctaaccaccgtggacaagtgggcgacggtgtctgcagggcggtagcgcggaaatggcctcaagccttccgtaacgcagcgacacctgtcggaaccgcaaaaaccgtcaagtgcgacgagacttacatcatccacgcggtgggtccaaattttaacaacacatctgaggctgagggggatcgtgacttggcggcggcgtaccgagccgtagcagcggagattaaccgactgtctataagtagtgtggcgattccgctgctttccacaggtatattcagtgcaggaaaagatagagtgcatcagtcgctttcgcaccttctagcggcaatggacactactgaagcacgggttaccatctactgccgcgataaaacgtgggagcaaaagatcaaaacagttctgcagaatcgctgcgccaccgaactggtgtccgatgagctacagcttgaagtcaatttgaccagagtccatccggatagtagcctggtgggacgtccagggtacagtaccactgatgggaccttatattcctatatggaaggtactaagttccaccaagcggctctggacatggccgagatcacgactttgtggccgagagttcaggatgcaaatgaacacatttgcatgtatgcactgggcgagacgatggataacatccgctctagatgccccgttgaggatagtgactcatcgactccaccgaagacggtcccatgcctgtgtcggtacgctatgacaccagagagagttacaagactacgaatgcatcacacaaaagatcttgtggtctgctcgtctttccagctaccgaagtaccgcatagctggtgtgcagcgagtaaagtgcgaaaaagtgatgctttttgatgcaactccaccggcctctgttagtcctgtgcaatacctgacgagtcacagcgaaactactgtaagcttgagctcgttctcaattacatccgacagtagctccctaagcacctttccggacctggagtcattagaagaacttggcaatgatccacagtccatgcggatggacgagtctgtcaaccaacaacccataccaacggcagaaccggttgttcaacccgtaccacctccgcgtcctaaacgtgccaggcgactggcggctgcacgtatgcaggtccaggtggaagtgcaccgcccacccgtcgtccaaaggacgaaaccggtcccagcaccgcgcaccagcttgcgccccgtccccgcgcctagaagctgtatgccaagaccagcagtcgagctgccctggccgcaggagaccgtcgacgtagagttcggggcgccgaccgaagaggatagtgaaatcacattcggagacttttctgcttcggagtgggagaccatcagtaactcatcctgactaggccgagcgggggcttatatcttctcatcggacgtcggtccagggcatctgcaacagaaatcagtgagacaacacgatctggaggtgccgattatggatcgcgtagtcgaggagaaagtctacccgccgaaatttgatgaggcaaaagagaaacagttgctcctaaaactgcagatgcacgccacagacgccaaccggagccggtaccaatcaaggaaagttgagaacatgaaagcaacgatcatcgaccggctgaaacagggcagcgcgtcttacatctcggctgaggccgataaagcagtcacataccatgttaaatatgctaagcctcggtactctgtgccggtgatgcaaagacttagcagtgcaaccaccgcagttgccgcttgcaatgaattcctggcccggaactaccctacagtggcgtcgtatcagatcaccgatgagtacgacgcttatttagatatggtggacgggtcagaaagctgtctagacagagcaaacttctgtccggcgaagttgcgctgctatccaaaacaccatgcataccatgtaccccagattagaagtgctgttccttcgccattccaaaacacgttgcagaatgtattagcggccgccactaaacgtaattgcaacgtcacccagatgcgtgaactaccaaccctggattcagccgtgtacaacgtggaatgtttccgcaagtacgcctgtaacaacgaatactgggaagagtttgctgctaaacctatcagaattacaacagagaatttgaccacttacgtgaccaaattaaaaggtggaaaggcagccgccctgtttgcaaagacgcataacttagttccactgcaggaagttccaatggatagattcgtcatggacatgaagcgcgatgtgaaggttacaccagggacgaagcacacagaggaacgaccaaaggtccaagtgattcaagctgccgagcctctggctaccgcctacctgtgtggaattcacagagaactggttcgccggctcaacgctgtgttgctacctaacatccataccctgtttgacatgtctgctgaagatttcgatgccatcatatcagagcacttcaagccaggggaccatgtcttggaaacggacatcgcttcttttgacaaaagccaggacgattcactggcactaacggggctgatgatactggaagatttgggcgtcgacaaccagttattggatcttatcgaggctgcgtttggtcagattaccagctgccaccttcctacagggactaggttcaaatttggtgctatgatgaagtcaggcatgtttcttacattgttcattaacaccgttttgaacattaccattgccagcagagtgctggaagccagattaactaactcagcctgtgccgcatttatcggcgacgacaacgtggttcacggagtcgtctccgataaactgatggcagatagatgcgccacatgggttaacatggaggttaaaataatagatgcagtcatgtgtgcaaagccaccgtatttctgtggcggctttttggtctatgatcatgtcacaaggatgtcgtgtcgaatagcggacccattaaaaaggttattcaaattgggcaaacctctgccggcagacgactgccaagatgaagaccgccgtagggcattgcacgacgaagttaaaaaatggtttagatcaggcttgggttcggagatcgaggtcgccctcgccaccagatacgaggtggaagggggttacaacctattgttggctatgtccacctttgcacacagtatgaagaatttttctgcattgaggggacccgtcatacacttgtacggcggtcctaaataggtgctctacacgacacctataccaccatggatttcctaccaacacaagtgttttacggcaggcgttggagaccacgaatgccgccacgcccttggaggccacgcccgcctacaattcaaagaccagatcaacaggcccgacaaatgcagcagttgattgccgcggtcagtacgcttgcccttaggcaaaacgctgctgcccctcagcgtggaagaaagaagcaaccacgtagaaagaaaccaaaaccacaacccgagaaacctaagaagcaggagcagaaaccgaagcaaaagaagacccctaagaagaagcccgggaggagggagcgcatgtgcatgaagattgagcacgattgcatctttgaggtcaagcacgaaggaaaggtcacaggttatgcctgccttgtcggcgacaaggtaatgaagccggcacacgtccctggagtgatagacaacatcgatctcgcacgtctatcgtataagaaatctagtaagtacgacctggaatgtgcgcaaataccggtggctatgaagtcagatgcatcgaaatacacccatgaaaaacccgaaggtcactataactggcactatggggccgtccagtacacaggaggcagattcacggtgcccacaggagtgggtaagcccggcgacagcggccggcccatctttgacaacaagggccgcgttgtcgcaatagtgctaggaggagctaacgaaggtgccaggaccgcgctctctgtcgtgacgtggaataaagacatggtcaccaagatcacaccggaaggcacagaggagtgggcagctccgacagtgacggctatgtgccttctggcgaatgtatccttcccttgtttccaaccgagctgctctccatgctgttatgaaaaggggcccgagccgacgctgagaatgctggaagagaacgtaaattcggagggatactatgaactgttgcatgctgccgtgtactgcaaaaacagctcgaggtcgaagaggagcaccgcgaaccactttaacgcatacaagctgactcgtccatacgtagcctactgtgcagactgcggaatgggccattcttgccacagtcccgccatgattgaaaacgtgcaggcggatgctacagacggcacgttaaagattcagttcgcttctcaaatcggcctaacgaaaacggacacgcatgatcacacaaagattagatatgcagaagggcatgatatagcggaggctgccaggtcaactcttaaggtccatagcagcagtgagtgcgcggtgacaggaacgatgggacacttcatcttggccaaatgtccaccgggcgaagtcattagtgtctcatttgttgattcaaaaaatgaacagcggacctgtcggatagcctaccgccatgaacagaggctaatagggcgtgaaagattcacggtgcgaccacatcacgggatcgagctaccttgcaccacttaccagctaacgaccgccgaaacctctgaagagatagacatgcacatgccgccggacgttccggacagaactattctttcccagcaatcaggaaacgtcaagataacggtcaacgggcggaccgttaagtacagctgctcatgtggttctaaaccatcaggcacaacaaccacagacaagactatcaatagctgcaccgttgacaaatgccaggcatatgtcacgagccatacgaaatggcaatttaactcacctttcgttccgcgcgcggagcaagcggagcgtaagggcaaggtgcacattccctttccacttatcaacaccacttgccgagtaccgttggctcccgaagcccttgtcagaagcggtaaacgagaagctacgctctcactgcacccgatacatcccacattgttaagttacagaacactgggacgcgagccagtttttgatgaacagtggatcaccacccagacggaggtaacaatcccagtaccagtggagggagtggagtatcggtggggcaaccataaaccgcaacgcttgtggtcacagttaacaactgaaggcagagcccatggctggccccatgagattatcgagtactactacgggctgcatcctacgacaaccattgtcgtggtggttgctgtctcagtagtggtgctcttgtcagttgccgcctccgtttatatgtgtgtagtggcacgcaacaagtgcctgacaccgtacgcactcacccccggggccgtcgtccctgtcactatcggggtgttgtgctgtgcaccgaaagcgcacgcagctagctttgctgaaggcatggcctatctgtgggataataaccagtcaatgttctggatggagttgactggacctttggctcttcttatcctgactacatgttgcgcccgatcactactttcctgctgcaagggctcttttttagtcgcagtgagcgtcgggagtgccgttgccagtgcttacgagcacacggcagtcattccaaatcaagtgggattcccgtataaggctcatgttgcacgcgaaggatacagcccgctgacactgcagatgcaggtgattgaaaccagtcttgagccaacgctcaacctggagtacatcacttgcgaatacaagacaaaggtcccatcaccgtacgtaaagtgctgcggtacagctgaatgccgcacgcaggacaagcccgaatacaagtgtgcggtgttcacgggcgtgtacccttttatgtggggaggtgcatactgtttctgcgattcggaaaacacacagatgagtgaagcctacgtggagcgcgctgacgtgtgcaaacacgactacgcagccgcctaccgcgcccacactgcttccctcagagccaaaatcaaggtagcatatggtaccgtgaatcagactgtcgaggcgtatgtgaacggtgaccatgccgtaacgattgccgggacgaagtttatctttggtccggtgtcaacggcctggacaccgttcgatactaaaatcgtggtctataaaggggaggtatacaaccaggactttcccccgtacggtgccgggcagcctggaagattcggagacatccagagtaggacgttggacagtaaggacctatacgctaatacgggccttaagctggcaagaccggcagccggcaacatccacgtcccctatacccagacaccatctggttttaaaacatggcaaaaagacagggactcaccgctaaacgctaaggcaccctttggatgtacaatccagacaaatccggtcagagcgatgaattgcgctgtcggcaacatacccgtttcgatggatatcgccgacagcgcgttcactagactgaccgatgcgcctataatatcagagctgctgtgcactgtatctacatgcacgcattcttcagacttcggcggagtcgctgtactttcttacaaggtggaaaaagctggcaggtgcgacgtccattcgcactcgaacgtcgcggtactccaggaagttttcatcgaggcagaaggtcgatcagtgatccacttttcgaccgcatcagccgccccttccttcatagtatccgtctgcagctcgcgtgccacgtgcacagcgaaatgtgaaccaccgaaagaccatgtggtcacctatccggcgaatcacaacgggataattttgccggacttatccagcactgcaatgacttgggcgcaacatcttgccggtggagttgggctattgatagctctggcggtgctaattctagtaatagttacttgcataactttgagaaggtgaatcataagtaccgtgtaatgcgggctactatatatgtaatcattattatattcttaactatattaatattcaattatcaaattcggtgccgtgcatgatgcggaccgatttattattcacatatgtaatccttattatatcatatcacatattcaatttcaaaatttctatacgcgtctttaatggcgcatataataatcacctataatttttcttatttttctttatatgtgccactatagggcacctactaaccatagaagtaattcattttgtttttaatatttcaaaaaaaaaaaaaaaaaaaaaaaaaaaaaaaaaaa

SEQ ID NO: 2 - DNA, 11462 bp, synthetic construct
  source 1..11462: mol_type = other DNA
atggcgggcaagtgacacttgttccgccagtcgtctctaagctcttcctctgcattgcaagagtttaccactcagtatgtcgaaagtctttgtagatatcgaggccgagagcccgtttttaaaatcactacagagagcgtttccagcatttgaagtggaagcacagcaggtcacaccaaatgaccatgctaacgccagagcattttcgcatctggctactaaattgatagagcaagagaccgaaaaagacacactcatcctggatatcggcagtgcgcctgccaggagaatgatgtctgagcacacgtaccactgtgtgtgcccaatgcgcagcgctgaggacccagagcgtctgctgtattatgccaggaagttggccaaggcatcaggagaagtcgttgacagaaacatcgcagcgaagatagacgacctgcagtcagtgatggcgaccccggacaatgagtcacggacattttgccttcacacagatcagacatgcaggactccagcagaggtggcagtctatcaggatgtttatgcagttcatgcaccgacttctttgtacttccaggcaatgaaaggagtacgcacagcgtactggattgggttcgatactaccccgttcatgttcgatacaatggccggggcttatccaacatatgcaaccaactgggccgacgaacaggtgttgaaagccaggaacataggactatgctccgcggcactgactgagggacaccttggcaagctatcaataatgaggaagaagagaatgaaaccgagtgaccagataatgttctcggtaggttccacattgtatacggaaagccgacgtctgctaaagagttggcatctaccatcagtgttccatctgaaaggacgacaatcgtatacgtgccggtgcgatacaatagtgtcatgtgagggctacgttgttaagaagataacgatgagcccaggggtattcggaaaaacgtcagggtacgccgtcacacatcacgcagaaggatttctggtatgtaaaatcactgacactatcgccggagaacgagtttcgttccccgtctgcacgtatgtgccatcaacgatctgtgaccaaatgacaggcattctggcgactgaggtcacgcctgaggatgcgcaaaaattgcttgtcggattaaaccagaggatcgtcgtgaatgggaggacccagaggaacactaatacaatgaagaactacctgcttccagtggtgtcacaagcatttagtaagtgggcgaaagagtatcggcttgaccaagaagatgagaaaaacatgggtatgcgcgaacgcaccctgacttgctgctgcctgtgggcctttaagatccataagaatcacaccatgtataagaagccggatacacagacgatcgtcagggtcccgtcggagtttaactccttcgtgatccctaacctgtggtcagcggggttgtcgatcggaattagacacaggatcaggcttcttttgcagtcaagacgtgccgagccgctcgttccatctatggatgctagtgaagccagagcagcagagaaggaggctgcagaagctaaggaggcagaagagacactggcagccctgccacctttgatcccgaccgctccgctgctcgacgacatccctgaagtggatgtagaggagctggagtttcgagcaggagccggtgttgttgaaacgcctcggaatgccctcaaggttacaccacaagacagagataccatggtaggtagctatcttgttctgtctccccagacagtcctgaaaagtgctaaactgcaggtactgcaccccttggcagagcaagtaaaaatcatcacccacaaaggccgtgcagggcgctaccaagtggatgcctatgacggccgtgttttaatccccacaggcgccgctatcccggtacccgatttccaggctctaagtgagagtgcgactatggtgtacaacgaacgtgagttcatcaatcgcaaattgtaccacatagccgtacatggggcggcactgaataccgatgaggagggttacgagaaagtgcgggccgaaaggacagatgccgaatacgtctttgacgtggatcgcaaacagtgcgttaagagggaggatgccgagggtctcgtgatgatcggagacctggtcaaccctccattccacgaatttgcgtatgaaggactgaaacgtagaccagcggctccatacaaaacaactgtggtcggagtcttcggcgtgcctggatctggaaaatcgggtataatcaaaagcctggtcacacgtgcagacttggtcaccagtggaaagagagaaaactgtcaggagatcatgcttgacgtcaagagatacagagacctggacatcactgcaaagacggtggattccgtattgttgaatggcgttaagcagactgtcgatgtcttgtacgtcgatgaagctttcgcttgccatgcagggacactgctagcgcttatcgccacggtacgaccacgtaagaaggttgtgctttgcggcgacccaaaacagtgcggtttcttcaacttgatgcaactacaggtcaactttaaccacaacatctgcaccgaggtgcatcataagagcatctccagaagatgcacgctaccaatcaccgccattgtctcaacattacattatgagggcaaaatgcgcaccactaacccatacaataaacctgttattattgacaccacaggccagactaaaccaaaccgcgaggacattgtgttaacatgtttccgcggttgggtcaaacagctgcaacttgactatcgtgggcacgaagtcatgacagccgctgcatcccaggggttgacccgaaagggcgtgtacgccgtccgaatgaaggtcaacgaaaacccactgtacgcacaatcatcggagcatgttaatgttctactgacacgcacagagggcagactagtgtggaagacactgtcaggagatccctggatcaaaactttgagcaacatcccaaaagggaatttcacagcaactttggaagattggcaacaagagcacgacgccattatgagggcaataacacaagaagcagcccctttggacgtgttccaaaataaggctaaggtgtgttgggccaagtgtttggtacccgttttggaaaccgcggggatcaggttgtcagccgccgattggagctcaattattttggccttcaaagaagacagagcttattcaccagaggttgcactgaatgagatctgcacaaaagtctacggggtggatctagacagcggcctgttttcggctccacgcgtgtcgctacactatactacaaatcattgggataactcgcctggaggaaggatgtacgggttttccgttgaggccgccaaccgcctagaacaacggcacccgttctacaggggacggtgggcttctgggcaggtgttggtcgcagaacgaagaactcagccgattgacatcacttgcaacctaatccccttcaaccggagactcccacacgcgttggtcacggaataccatccagttaaaggagaaagagtggagtggcttgtgaataagatcccaggctatcacttactactggtcagcgagtataacctcatactgcctagaaggaaggtaacatggatcgccccgccgactgtgacaggagccgatttaacccatgacttggatctaggactaccgcctaatgctggcaggtatgacctagtcttcgtcaacatgcatacaccgtacaggctccatcactaccaacaatgcgtcgatcacgccatgaaattacagatgctgggcggcgacgcactctacctgttgaaacccgggggaagcctccttttgagagcctacggttatgccgatagaacgagtgaggctgtggtgacggctctcgctcgtcggttctcgtccttcagagcggtcagacctccatgtgtgactagtaacaccgaggtgttcttgctgttctcgaactttgacaatggtagaagaacagtaaccctgcatcctacaaatggtaagctatcatcaatctatgcaggtacagtgctgcaggcggctggctgtgctcccgcttacactgtcaaaagggcagacatcgcgaccgccattgaggatgcggtggtcaatgcagctaaccaccgtggacaagtgggcgacggtgtctgcagggcggtagcacggaaatggcctcaagccttccgtaacgcagcgacacctgtcggaaccgcaaaaaccgtcaagtgcgacgagacttacatcatccatgcggtgggtccaaattttaacaacacatctgaggctgagggggatcgtgacttggcggcggcgtacagagccgtagcagcggagattaaccgactgtctataagtagtgtggcgattccactgctttccacaggtatattcagtgcaggaaaagatagagtgcatcagtcgctttcgcaccttctagcggcaatggacactactgaagcacgggttaccatctactgccgcgataaaacgtgggagcaaaagatcaaaacagttctgcagaatcgctgcgccactgaactggtgtctgatgagctacagcttgaagtcaatttgaccagagtccatccggatagtagcctggtgggacgtccagggtacagtaccactgatgggaccttatattcctatatggaaggtactaagttccaccaagcggctctggacatggctgagatcacgactttgtggccgagagttcaggatgcaaatgaacacatttgcatgtatgcactgggcgagacgatggataacatccgctctagatgcccagttgaggatagtgactcatcgactccaccaaagacggtcccatgcctgtgtcggtacgctatgacaccagagagagttacaagactacgaatgcatcacacaaaagatcttgtggtctgctcgtctttccagctaccgaagtaccgcatagctggtgtgcagcgagtaaagtgcgaaaaagtgatgctttttgatgcaactccaccggcctctgttagtcctgtgcaatacctgacgagtcacagtgaaactactgtaagcttgagctcgttctcaattacatctgacagtagctccctaagcacctttccggacctggagtcattagaagaacttggcaatgatccacagtccatgcggatggacgagtctgtcaaccaacaacccataccaacggcagaaccggttgttcaacccgtaccacctccgcgtcctaaacgtgccaggcgactggcggctgcacgtatgcaggtccaggtggaagtgcaccgcccacccgtcgtccaaaggacgaaaccggtcccagcaccgcgcaccagcttgcgccccgtccccgcgcctagaagctgtatgccaagaccagcagttgagctgccctggccgcaggagaccgtcgacgtagagttcggggcgccgaccgaagaggatagtgaaatcacattcggagacttttctgcttcggagtgggagaccatcagtaactcatcctgactaggccgagcgggggcttatatcttctcatcagacgtcggtccagggcatctgcaacagaaatcagtgagacaacacgatctggaggtgccgattatggatcgcgtagttgaggagaaagtctacccgccgaaatttgatgaggcaaaagagaaacagttgctcctaaaactgcagatgcatgccacagatgccaaccggagccggtaccaatcaaggaaagttgagaacatgaaagcaacgatcattgaccggctgaaacagggcagcgcatcttacatctcggctgaggccgataaagcagtcacataccatgttaaatatgctaagcctcggtactctgtgccggtgatgcaaagacttagcagtgcaaccaccgcagttgcagcttgcaatgaattcctggcccggaactaccctacagtggcgtcgtatcagatcaccgatgagtatgacgcttatttagatatggtggacgggtcagaaagctgtctagacagagcaaacttctgtccggcgaagttgcgctgctatccaaaacaccatgcataccatgtaccccagattagaagtgctgttccttcgccattccaaaacacgttgcagaatgtattagcggccgccactaaacgtaattgcaacgtcacccagatgcgtgaactaccaaccctggattcagccgtgtacaacgtggaatgtttccgcaagtacgcctgtaacaacgaatactgggaagagtttgctgctaaacctatcagaattacaacagagaatttgaccacttatgtgaccaaattaaaaggtggaaaggcagccgccctgtttgcaaagacgcataacttagttccactgcaggaagttccaatggatagatttgtcatggacatgaagcgcgatgtgaaggttacaccagggacgaagcacacagaggaacgaccaaaggtccaagtgattcaagctgccgagcctctggctaccgcctacctgtgtggaattcacagagaactggttcgccggctcaatgctgtgttgctacctaacatccataccctgtttgacatgtctgctgaagattttgatgccatcatatcagagcacttcaagccaggggaccatgtcttggaaacggacatcgcttcttttgacaaaagccaggacgattcactggcactaacagggctgatgatactggaagatttgggcgtcgacaaccagttattggatcttatcgaggctgcgtttggtcagattaccagctgccaccttcctacagggactaggttcaaatttggtgctatgatgaagtcaggcatgtttcttacattgttcattaacaccgttttgaacattaccattgccagcagagtgctggaagccagattaactaactcagcctgtgccgcatttatcggcgacgacaacgtggttcacggagtcgtctccgataaactgatggcagatagatgtgccacatgggttaacatggaggttaaaataatagatgcagtcatgtgtgcaaagccaccttatttctgtggaggctttttggtctatgatcatgtcacaaggatgtcgtgtcgaatagcggacccattaaaaaggttattcaaattgggcaaacctctgccggcagatgactgccaagatgaagaccgccgtagggcattgcacgacgaagttaaaaaatggtttagatcaggcttgggttcggagatcgaggtcgccctcgccaccagatacgaggtggaagggggttacaacctattgttggctatgtccacctttgcacacagtatgaagaatttttctgcattgaggggacccgtcatacacttgtacggcggtcctaaataggtgctctacacgacacctataccaccatggatttcctaccaacacaagtgttttacggcaggcgttggagaccacgaatgccgccacgcccttggaggccacgcccgcctacaattcaaagaccagatcaacaggcccgacaaatgcagcagttgattgccgcggtcagtacgcttgcccttaggcaaaacgctgctgcccctcagcgtggaagaaagaagcaaccacgtagaaagaaaccaaaaccacaacccgagaaacctaagaagcaggagcagaaaccgaagcaaaagaagacccctaagaagaagcccgggaggagggagcgcatgtgcatgaagattgagcacgattgcatctttgaggtcaagcacgaaggaaaggtcacaggttatgcctgccttgtcggcgacaaggtaatgaagccggcacacgtccctggagtgatagacaacatcgatctcgcacgtctatcgtataagaaatctagtaagtacgacctggaatgtgcgcaaataccggtggctatgaagtcagatgcatcgaaatacacccatgaaaaacccgaaggtcactataactggcactatggggccgtccagtacacaggaggcagattcacggtgcccacaggagtgggtaagcccggcgacagcggccggcccatctttgacaacaagggccgcgttgtcgcaatagtgctaggaggagctaacgaaggtgccaggaccgcgctctctgtcgtgacgtggaataaagacatggtcaccaagatcacaccggaaggcacagaggagtgggcagctccgacagtgacggctatgtgccttctggcgaatgtatccttcccttgtttccaaccgagctgctctccatgctgttatgaaaaggggcccgagccgacgctgagaatgctggaagagaacgtaaattcggagggatactatgaactgttgcatgctgccgtgtactgcaaaaacagctcgaggtcgaagaggagcaccgcgaaccactttaacgcatacaagctgactcgtccatacgtagcctactgtgcagactgcggaatgggccattcttgccacagtcccgccatgattgaaaacgtgcaggcggatgctacagacggcacgttaaagattcagttcgcttctcaaatcggcctaacgaaaacggacacgcatgatcacacaaagattagatatgcagaagggcatgatatagcggaggctgccaggtcaactcttaaggtccatagcagcagtgagtgcgcggtgacaggaacgatgggacacttcatcttggccaaatgtccaccgggcgaagtcattagtgtctcatttgttgattcaaaaaatgaacagcggacctgtcggatagcctaccgccatgaacagaggctaatagggcgtgaaagattcacggtgcgaccacatcacgggatcgagctaccttgcaccacttaccagctaacgaccgccgaaacctctgaagagatagacatgcacatgccgccggacgttccggacagaactattctttcccagcaatcaggaaacgtcaagataacggtcaacgggcggaccgttaagtacagctgctcatgtggttctaaaccatcaggcacaacaaccacagacaagactatcaatagctgcaccgttgacaaatgccaggcatatgtcacgagccatacgaaatggcaatttaactcacctttcgttccgcgcgcggagcaagcggagcgtaagggcaaggtgcacattccctttccacttatcaacaccacttgccgagtaccgttggctcccgaagcccttgtcagaagcggtaaacgagaagctacgctctcactgcacccgatacatcccacattgttaagttacagaacactgggacgcgagccagtttttgatgaacagtggatcaccacccagacggaggtaacaatcccagtaccagtggagggagtggagtatcggtggggcaaccataaaccgcaacgcttgtggtcacagttaacaactgaaggcagagcccatggctggccccatgagattatcgagtactactacgggctgcatcctacgacaaccattgtcgtggtggttgctgtctcagtagtggtgctcttgtcagttgccgcctccgtttatatgtgtgtagtggcacgcaacaagtgcctgacaccgtacgcactcacccccggggccgtcgtccctgtcactatcggggtgttgtgctgtgcaccgaaagcgcacgcagctagctttgctgaaggcatggcctatctgtgggataataaccagtcaatgttctggatggagttgactggacctttggctcttcttatcctgactacatgttgcgcccgatcactactttcctgctgcaagggctcttttttagtcgcagtgagcgtcgggagtgccgttgccagtgcttacgagcacacggcagtcattccaaatcaagtgggattcccgtataaggctcatgttgcacgcgaaggatacagcccgctgacactgcagatgcaggtgattgaaaccagtcttgagccaacgctcaacctggagtacatcacttgcgaatacaagacaaaggtcccatcaccgtacgtaaagtgctgcggtacagctgaatgccgcacgcaggacaagcccgaatacaagtgtgcggtgttcacgggcgtgtacccttttatgtggggaggtgcatactgtttctgcgattcggaaaacacacagatgagtgaagcctacgtggagcgcgctgacgtgtgcaaacacgactacgcagccgcctaccgcgcccacactgcttccctcagagccaaaatcaaggtagcatatggtaccgtgaatcagactgtcgaggcgtatgtgaacggtgaccatgccgtaacgattgccgggacgaagtttatctttggtccggtgtcaacggcctggacaccgttcgatactaaaatcgtggtctataaaggggaggtatacaaccaggactttcccccgtacggtgccgggcagcctggaagattcggagacatccagagtaggacgttggacagtaaggacctatacgctaatacgggccttaagctggcaagaccggcagccggcaacatccacgtcccctatacccagacaccatctggttttaaaacatggcaaaaagacagggactcaccgctaaacgctaaggcaccctttggatgtacaatccagacaaatccggtcagagcgatgaattgcgctgtcggcaacatacccgtttcgatggatatcgccgacagcgcgttcactagactgaccgatgcgcctataatatcagagctgctgtgcactgtatctacatgcacgcattcttcagacttcggcggagtcgctgtactttcttacaaggtggaaaaagctggcaggtgcgacgtccattcgcactcgaacgtcgcggtactccaggaagttttcatcgaggcagaaggtcgatcagtgatccacttttcgaccgcatcagccgccccttccttcatagtatccgtctgcagctcgcgtgccacgtgcacagcgaaatgtgaaccaccgaaagaccatgtggtcacctatccggcgaatcacaacgggataattttgccggacttatccagcactgcaatgacttgggcgcaacatcttgccggtggagttgggctattgatagctctggcggtgctaattctagtaatagttacttgcataactttgagaaggtgaatcataagtaccgtgtaatgcgggctactatatatgtaatcattattatattcttaactatattaatattcaattatcaaattcggtgccgtgcatgatgcggaccgatttattattcacatatgtaatccttattatatcatatcacatattcaatttcaaaatttctatacgcgtctttaatggcgcatataataatcacctataatttttcttatttttctttatatgtgccactatagggcacctactaaccatagaagtaattcattttgtttttaatatttcaaaaaaaaaaaaaaaaaaaaaaaaaaaaaaaaaaa

SEQ ID NO: 3 - DNA, 11462 bp, synthetic construct
  source 1..11462: mol_type = other DNA
atggcgggcaagtgacacttgttccgccagtcgtctctaagctcttcctctgcattgcaagagtttaccactcagtatgtcgaaagtctttgtagatatcgaggccgagagcccgtttttaaaatcactacagagagcgtttccagcatttgaagtggaagcacagcaggtcacaccaaatgaccatgctaacgccagagcattttcgcatctggctactaaattgatagagcaagagaccgaaaaagacacactcatcctggatatcggcagcgcgcctgccaggagaatgatgtctgagcacacgtaccactgtgtgtgcccaatgcgcagcgctgaggacccagagcgtctgctgtattatgccaggaagttggccaaggcatcaggcgaagtcgttgacagaaacatcgcagcgaagatagacgacctgcagtcagtgatggcgaccccggacaatgagtcacggacattttgccttcacacagatcagacatgcaggactccagcagaggtggcggtctatcaggatgtttatgcagttcacgcaccgacttctttgtacttccaggcgatgaaaggagtacgcacagcgtactggattgggttcgatactaccccgttcatgttcgatacaatggccggggcttatccaacatatgcaaccaactgggccgacgaacaggtgttgaaagccaggaacataggactatgctccgcggcactgactgagggacaccttggcaagctatcgataatgaggaagaagagaatgaaaccgagtgaccagataatgttctcggtaggttccacattgtatacggaaagccgacgtctgctaaagagttggcatctaccgtcagtgttccatctgaaaggacgacaatcgtatacgtgccggtgcgatacaatagtgtcatgtgagggctacgttgttaagaagataacgatgagcccaggggtattcggaaaaacgtcagggtacgccgtcacacatcacgcagaaggatttctggtatgtaaaatcaccgacactatcgcgggagaacgagtttcgttccccgtctgcacgtatgtgccgtcgacgatctgtgaccaaatgacaggcattctggcgaccgaggtcacgcccgaggacgcgcaaaaattgcttgtcggattaaaccagaggatcgtcgtgaacgggaggacccagaggaacactaatacaatgaagaactacctgcttccagtggtgtcacaagcatttagtaagtgggcgaaagagtatcggcttgaccaagaagatgagaaaaacatgggtatgcgcgaacgcaccctgacgtgctgctgcctgtgggcctttaagatccataagaatcacaccatgtataagaagccggatacacagacgatcgtcagggtcccgtcggagtttaactccttcgtgatccctaacctgtggtcagcggggttgtcgatcggaattagacacaggatcaggcttcttttgcagtcaagacgtgcggagccgctcgttccatctatggatgctagtgaagccagagcggcagagaaggaggctgcagaagctaaggaggcagaagagacactggcagccctgccacctttgatcccgaccgctccgctgctcgacgacatccctgaagtggatgtagaggagctggagtttcgagcaggagccggcgttgtcgaaacgcctcggaacgccctcaaggttacaccacaagacagagataccatggtaggtagctatctcgttctgtctccccagacagtcctgaaaagtgctaaactgcaggtactgcaccccttggcagagcaagtaaaaatcatcacccacaaaggccgtgcagggcgctaccaagtggatgcctatgacggccgcgttttaatcccgacaggcgcggctatcccggtacccgatttccaggctctaagtgagagtgcgactatggtgtacaacgaacgtgagttcatcaatcgcaaattgtaccacatagccgtacatggggcggcactgaataccgatgaggagggttacgagaaagtgcgggccgaaaggacagatgccgaatacgtctttgacgtggatcgcaaacagtgcgttaagagggaggacgccgagggtctcgtgatgatcggagacctggtcaaccctccattccacgaatttgcgtacgaaggactgaaacgtagaccggcggctccgtacaaaacaaccgtggtcggagtcttcggcgtgcctggatctggaaaatcgggtataatcaaaagcctggtcacacgcgcggacttggtcaccagtggaaagagagaaaactgtcaggagatcatgctcgacgtcaagagatacagagacctggacatcactgcgaagacggtggattccgtattgttgaatggcgttaagcagactgtcgacgtcttgtacgtcgatgaagctttcgcttgccatgcagggacactgctagcgcttatcgccacggtacgaccacgtaagaaggtcgtgctttgcggcgacccgaaacagtgcggtttcttcaacttgatgcaactacaggtcaactttaaccacaacatctgcaccgaggtgcatcataagagcatctccagaagatgcacgctaccaatcaccgccattgtctcaacattacattatgagggcaaaatgcgcaccactaacccatacaataaacccgttattattgacaccacaggccagactaaaccaaaccgcgaggacattgtgttaacgtgtttccgcggttgggtcaaacagctgcaacttgactatcgtgggcacgaagtcatgacagccgctgcatcccaggggttgacccgaaagggcgtgtacgccgtccgaatgaaggtcaacgaaaacccactgtacgcacaatcatcggagcatgttaatgttctactgacacgcacagagggcagactagtgtggaagacactgtcgggagatccctggatcaaaactttgagcaacatcccaaaagggaatttcacagcaactttggaagattggcaacaagagcacgacgccattatgagggcaataacacaagaagcagcccctttggacgtgttccaaaataaggctaaggtgtgttgggccaagtgtttggtacccgttttggaaaccgcggggatcaggttgtcagccgccgattggagctcaattattttggccttcaaagaagacagagcttattcaccagaggttgcactgaatgagatctgcacaaaagtctacggggtggatctagacagcggcctgttttcggctccacgcgtgtcgctacactatactacaaatcattgggataactcgcctggaggaaggatgtacgggttttccgtcgaggccgccaaccgcctagaacaacggcacccgttctacaggggacggtgggcttctgggcaggtgttggtcgcagaacgaagaactcagccgattgacatcacttgcaacctaatccccttcaaccggagactcccacacgcgttggtcacggaataccatccagttaaaggagaaagagtggagtggcttgtgaataagatcccaggctatcacttactactggtcagcgagtataacctcatactgcctagaaggaaggtaacgtggatcgccccgccgactgtgacaggagccgatttaacccacgacttggatctaggactaccgcctaacgctggcaggtatgacctagtcttcgtcaacatgcatacaccgtacaggctccatcactaccaacaatgcgtcgatcacgccatgaaattacagatgctgggcggcgacgcactctacctgttgaaacccgggggaagcctccttttgagagcctacggttacgccgatagaacgagcgaggctgtggtgacggctctcgctcgtcggttctcgtccttcagagcggtcagacctccatgtgtgactagtaacaccgaggtgttcttgctgttctcgaactttgacaacggtagacgaacagtaaccctgcatcctacaaacggtaagctatcatcgatctatgcaggtacagtgctgcaggcggccggctgcgctcccgcttacaccgtcaaaagggcagacatcgcgaccgccattgaggatgcggtggtcaacgcagctaaccaccgtggacaagtgggcgacggtgtctgcagggcggtagcgcggaaatggcctcaagccttccgtaacgcagcgacacctgtcggaaccgcaaaaaccgtcaagtgcgacgagacttacatcatccacgcggtgggtccaaattttaacaacacatctgaggctgagggggatcgtgacttggcggcggcgtaccgagccgtagcagcggagattaaccgactgtctataagtagtgtggcgattccgctgctttccacaggtatattcagtgcaggaaaagatagagtgcatcagtcgctttcgcaccttctagcggcaatggacactactgaagcacgggttaccatctactgccgcgataaaacgtgggagcaaaagatcaaaacagttctgcagaatcgctgcgccaccgaactggtgtccgatgagctacagcttgaagtcaatttgaccagagtccatccggatagtagcctggtgggacgtccagggtacagtaccactgatgggaccttatattcctatatggaaggtactaagttccaccaagcggctctggacatggccgagatcacgactttgtggccgagagttcaggatgcaaatgaacacatttgcatgtatgcactgggcgagacgatggataacatccgctctagatgccccgttgaggatagtgactcatcgactccaccgaagacggtcccatgcctgtgtcggtacgctatgacaccagagagagttacaagactacgaatgcatcacacaaaagatcttgtggtctgctcgtctttccagctaccgaagtaccgcatagctggtgtgcagcgagtaaagtgcgaaaaagtgatgctttttgatgcaactccaccggcctctgttagtcctgtgcaatacctgacgagtcacagcgaaactactgtaagcttgagctcgttctcaattacatccgacagtagctccctaagcacctttccggacctggagtcattagaagaacttggcaatgatccacagtccatgcggatggacgagtctgtcaaccaacaacccataccaacggcagaaccggttgttcaacccgtaccacctccgcgtcctaaacgtgccaggcgactggcggctgcacgtatgcaggtccaggtggaagtgcaccgcccacccgtcgtccaaaggacgaaaccggtcccagcaccgcgcaccagcttgcgccccgtccccgcgcctagaagctgtatgccaagaccagcagtcgagctgccctggccgcaggagaccgtcgacgtagagttcggggcgccgaccgaagaggatagtgaaatcacattcggagacttttctgcttcggagtgggagaccatcagtaactcatcctgactaggccgagcgggggcttatatcttctcatcggacgtcggtccagggcatctgcaacagaaatcagtgagacaacacgatctggaggtgccgattatggatcgcgtagtcgaggagaaagtctacccgccgaaatttgatgaggcaaaagagaaacagttgctcctaaaactgcagatgcacgccacagacgccaaccggagccggtaccaatcaaggaaagttgagaacatgaaagcaacgatcatcgaccggctgaaacagggcagcgcgtcttacatctcggctgaggccgataaagcagtcacataccatgttaaatatgctaagcctcggtactctgtgccggtgatgcaaagacttagcagtgcaaccaccgcagttgccgcttgcaatgaattcctggcccggaactaccctacagtggcgtcgtatcagatcaccgatgagtacgacgcttatttagatatggtggacgggtcagaaagctgtctagacagagcaaacttctgtccggcgaagttgcgctgctatccaaaacaccatgcataccatgtaccccagattagaagtgctgttccttcgccattccaaaacacgttgcagaatgtattagcggccgccactaaacgtaattgcaacgtcacccagatgcgtgaactaccaaccctggattcagccgtgtacaacgtggaatgtttccgcaagtacgcctgtaacaacgaatactgggaagagtttgctgctaaacctatcagaattacaacagagaatttgaccacttacgtgaccaaattaaaaggtggaaaggcagccgccctgtttgcaaagacgcataacttagttccactgcaggaagttccaatggatagattcgtcatggacatgaagcgcgatgtgaaggttacaccagggacgaagcacacagaggaacgaccaaaggtccaagtgattcaagctgccgagcctctggctaccgcctacctgtgtggaattcacagagaactggttcgccggctcaacgctgtgttgctacctaacatccataccctgtttgacatgtctgctgaagatttcgatgccatcatatcagagcacttcaagccaggggaccatgtcttggaaacggacatcgcttcttttgacaaaagccaggacgattcactggcactaacggggctgatgatactggaagatttgggcgtcgacaaccagttattggatcttatcgaggctgcgtttggtcagattaccagctgccaccttcctacagggactaggttcaaatttggtgctatgatgaagtcaggcatgtttcttacattgttcattaacaccgttttgaacattaccattgccagcagagtgctggaagccagattaactaactcagcctgtgccgcatttatcggcgacgacaacgtggttcacggagtcgtctccgataaactgatggcagatagatgcgccacatgggttaacatggaggttaaaataatagatgcagtcatgtgtgcaaagccaccgtatttctgtggcggctttttggtctatgatcatgtcacaaggatgtcgtgtcgaatagcggacccattaaaaaggttattcaaattgggcaaacctctgccggcagacgactgccaagatgaagaccgccgtagggcattgcacgacgaagttaaaaaatggtttagatcaggcttgggttcggagatcgaggtcgccctcgccaccagatacgaggtggaagggggttacaacctattgttggctatgtccacctttgcacacagtatgaagaatttttctgcattgaggggacccgtcatacacttgtacggcggtcctaaataggtgctctacacgacacctataccaccatggatttcctaccaacacaagtgttttatggcaggcgttggagaccacgaatgccaccacgcccttggaggccacgcccgcctacaattcaaagaccagatcaacaggcccgacaaatgcagcagttgattgctgcggtcagtacgcttgcccttaggcaaaacgctgctgcccctcagcgtggaagaaagaagcaaccacgtagaaagaaaccaaaaccacaacccgagaaacctaagaagcaggagcagaaaccgaagcaaaagaagacccctaagaagaagcccgggaggagggagcgcatgtgcatgaagattgagcacgattgcatctttgaggtcaagcacgaaggaaaggtcacaggttatgcctgccttgtcggtgacaaggtaatgaagccggcacacgtccctggagtgatagacaacatcgatctcgcacgtctatcgtataagaaatctagtaagtatgacctggaatgtgcgcaaataccggtggctatgaagtcagatgcatcgaaatacacccatgaaaaacccgaaggtcactataactggcactatggggctgtccagtacacaggaggcagattcacggtgcccacaggagtgggtaagcctggcgacagcggccggcccatctttgacaacaagggccgcgttgtcgcaatagtgctaggaggagctaatgaaggtgccaggaccgcgctctctgtcgtgacgtggaataaagacatggtcaccaagatcacaccggaaggcacagaggagtgggcagctccgacagtgacagctatgtgccttctggcgaatgtatccttcccttgtttccaaccgagctgctctccatgctgttatgaaaaggggcctgagccgacgctgagaatgctggaagagaacgtaaattcagagggatactatgaactgttgcatgctgccgtgtactgcaaaaacagctcgaggtcgaagaggagcactgcaaaccactttaacgcatacaagctgactcgtccatacgtagcctactgtgcagactgtggaatgggccattcttgccacagtcccgccatgattgaaaatgtgcaggcggatgctacagacggcacgttaaagattcagttcgcttctcaaattggcctaacgaaaacggacacgcatgatcacacaaagattagatatgcagaagggcatgatatagcggaggctgccaggtcaactcttaaggtccatagcagcagtgagtgcgcggtgacaggaacgatgggacacttcatcttggccaaatgtccaccaggcgaagtcattagtgtctcatttgttgattcaaaaaatgaacagcggacctgtcggatagcctaccgccatgaacagaggctaatagggcgtgaaagattcacggtgcgaccacatcacgggatcgagctaccttgcaccacttaccagctaacgaccgccgaaacctctgaagagatagacatgcacatgccgccagacgttccggacagaactattctttcccagcaatcaggaaacgtcaagataacggtcaatgggcggaccgttaagtacagctgctcatgtggttctaaaccatcaggcacaacaaccacagacaagactatcaatagctgcaccgttgacaaatgccaggcatatgtcacgagccatacgaaatggcaatttaactcacctttcgttccgcgtgcggagcaagcggagcgtaagggcaaggtgcacattccctttccacttatcaacaccacttgccgagtaccgttggctcctgaagcccttgtcagaagcggtaaacgagaagctacactctcactgcacccgatacatcccacattgttaagttacagaacactgggacgcgagccagtttttgatgaacagtggatcaccacccagacggaggtaacaatcccagtaccagtggagggagtggagtatcggtggggcaaccataaaccgcaacgcttgtggtcacagttaacaactgaaggcagagcccatggctggccccatgagattattgagtactactacgggctgcatcctacgacaaccattgtcgtggtggttgctgtctcagtagtggtgctcttgtcagttgccgcctctgtttatatgtgtgtagtggcacgcaacaagtgcctgacaccatatgcactcaccccgggggccgttgtccctgtcactatcggggtgttgtgctgtgcaccgaaagcgcatgcagctagctttgctgaaggcatggcctatctgtgggataataaccagtcaatgttctggatggagttgactggacctttggctcttcttatcctgactacatgttgcgcccgatcactactttcctgctgcaagggctcttttttagtcgcagtgagcgtcgggagtgccgttgccagtgcttacgagcacacggcagtcattccaaatcaagtgggattcccgtataaggctcatgttgcacgtgaaggatacagcccgctgacactgcagatgcaggtgattgaaaccagtcttgagccaacgctcaacctggagtacatcacttgtgaatacaagacaaaggtcccatcaccgtacgtaaagtgctgtggtacagctgaatgccgcacgcaggacaagcccgaatacaagtgtgcggtgttcacgggcgtgtacccttttatgtggggaggtgcatactgtttctgtgattcggaaaacacacagatgagtgaagcctacgtggagcgcgctgacgtgtgcaaacatgactatgcagccgcctaccgcgcccacactgcttccctcagagccaaaatcaaggtagcatatggtactgtgaatcagactgtcgaggcgtatgtgaacggtgaccatgccgtaacgattgccgggacgaagtttatctttggtccggtgtcaacggcctggacaccgttcgatactaaaatcgtggtctataaaggggaggtatacaaccaggactttcccccgtatggtgccgggcagcctggaagatttggagacatccagagtaggacgttggacagtaaggacctatatgctaatacgggccttaagctggcaagaccagcagccggcaacatccacgtcccctatacccagacaccatctggttttaaaacatggcaaaaagacagggactcaccgctaaacgctaaggcaccctttggatgtacaatccagacaaatccggtcagagcgatgaattgcgctgtcggcaacatacccgtttcgatggatatcgccgacagcgcattcactagactgactgatgcgcctataatatcagagctgctgtgcactgtatctacatgcacgcattcttcagacttcggtggagtcgctgtactttcttacaaggtggaaaaagctggcaggtgcgacgtccattcgcactcgaacgttgcggtactccaggaagttttcatcgaggcagaaggtcgatcagtgatccacttttcgaccgcatcagccgccccttccttcatagtatccgtctgcagctcacgtgccacgtgcacagcgaaatgtgaaccaccgaaagaccatgtggtcacctatccggcgaatcacaacgggataattttgccggacttatccagcactgcaatgacttgggcgcaacatcttgccggtggagttgggctattgatagctctggcggtgctaattctagtaatagttacttgcataactttgagaaggtgaatcataagtaccgtgtaatgcgggctactatatatgtaatcattattatattcttaactatattaatattcaattatcaaattcggtgccgtgcatgatgcggaccgatttattattcacatatgtaatccttattatatcatatcacatattcaatttcaaaatttctatacgcgtctttaatggcgcatataataatcacctataatttttcttatttttctttatatgtgccactatagggcacctactaaccatagaagtaattcattttgtttttaatatttcaaaaaaaaaaaaaaaaaaaaaaaaaaaaaaaaaaa